(19) United States
(12) Patent Application Publication
(10) Pub. No.: US 2026/0272840 A1
(43) Pub. Date: Sep. 17, 2026

(54) FORMATION OF CO-CRYSTALS

(71) Applicant: Indicatrix Crystallography Ltd., Newcastle upon Tyne, Tyne and Wear (GB)

(72) Inventors: Jessica P. METHERALL, Newcastle upon Tyne (GB); Lina Mardiana, Newcastle upon Tyne (GB); Michael R. PROBERT, Newcastle upon Tyne (GB); Michael J. Hall, Newcastle upon Tyne (GB)

(73) Assignee: Indicatrix Crystallography Ltd., Newcastle upon Tyne, Tyne and Wear (GB)

(21) Appl. No.: 19/471,207

(22) PCT Filed: Mar. 28, 2024
(86) PCT No.: PCT/GB2024/050875

§ 371 Date: Sep. 30, 2025

(30) Foreign Application Priority Data
Mar. 31, 2023 (GB) 2304851.5

(51) Int. Cl. A61K 9/14; A61K 31/4409; A61K 31/444; A61K 31/455; A61K 31/522; A61K 31/55; A61K 31/60

(52) U.S. Cl. CPC A61K 9/145; A61K 31/4409; A61K 31/444; A61K 31/455; A61K 31/522; A61K 31/55; A61K 31/60

(57) ABSTRACT

The invention relates to a method of forming a co-crystal, the method comprising forming a droplet comprising a first liquid and a second liquid; wherein the first liquid comprises a chemical molecule, or a salt thereof, and a co-former, or a salt thereof, and the second liquid is an oil. The invention also relates to a method of screening for co-crystals of a chemical molecule, or salt thereof, the method comprising said method of forming a co-crystal.

This invention relates to a method of forming co-crystals of chemical molecules. The methods are effective even when only very small amounts of a compound are available and can be used to screen for co-crystals of chemical molecules and to provide crystals for structural determination of unknown molecules by single crystal X-ray crystallography.

### BACKGROUND

Many chemical molecules are capable of packing together efficiently into a crystalline state. These systems often display multiple forms, including salts, solvates, co-crystals and polymorphic phases that differ only by the relative three dimensional (3D) arrangement of the molecules and their constituent atoms in space. Molecules can also pack in the crystalline state in multiple combinations of the above, including but not exclusively co-crystal solvates, co-crystal polymorphs and co-crystal salts.

Once suitable crystals can be obtained, considerable information about both the atomic connectivity and the three dimensional arrangement of the molecules within the crystal can be determined by a range of analytical techniques, including but not exclusively Raman spectroscopy, X-ray powder diffraction (PXRD) and single crystal X-ray diffraction (SCXRD).

Additionally, many molecular systems can be crystallised as co-crystals or salts where one or more molecules is crystallised with one or more co-formers. In the case of molecules and co-formers of significantly different pKa to allow charge transfer, these are typically known as salts. In the case of molecules and co-formers of similar pKa where no charge transfer occurs, these are typically known as co-crystals. Where the crystal includes one or more solvate molecules, these are typically known as solvates. Where the crystal includes molecules of water, these are typically known as hydrates. The resulting systems are then crystalline forms containing both the molecule(s) and co-former(s), and/or solvent, and/or water.

Co-crystals occur when two or more chemical molecules pack together in a crystalline state. Co-crystals can contain two or more molecules, and can display multiple forms including salts, solvates, and polymorphic phases that differ only by the relative three dimensional (3D) arrangement of the molecules and their constituent atoms in space.

The creation of co-crystals, their salts, solvates, and polymorphs, can result in changes in the physical properties of the crystalline structure, including but not limited to melting point, mechanical strength, compressibility, stability, dissolution rate, bioavailability and formulation granulation. The particular polymorphic phase of organic molecules can result in different physical properties (e.g. solubility) of the resulting solid form. This can provide benefits in the pharmaceutical industry, e.g. a readily formed particular polymorphic form of a co-crystal containing an active pharmaceutical ingredient (API) can simplify regulatory processes, but it can also cause problems, e.g. the unexpected appearance of a stable polymorphic phase of a co-crystal containing an API can cause significant problems requiring re-formulation.

Co-crystals may include co-former(s) that have no, or minimal, bioactivity. Alternatively, co-crystals may include a co-former(s) that have bioactivity, such as a second drug molecule or a prodrug, allowing the co-crystal to be used in the formulation of a drug which has a combination of bioactivities.

Modern SCXRD remains the analytical technique of choice for high resolution structural analysis (<0.9 Å) allowing access to absolute stereochemical determination, through anomalous dispersion measurements and to the molecular/atomic arrangement within the co-crystals themselves along with all intermolecular interactions and packing energies. However, SCXRD is not ubiquitously employed as an analysis method for co-crystals, due to the technical difficulties associated with the co-crystallisation of the analyte. The required quantities of material and experimental time constraints often restrict the simultaneous exploration of a two of more molecules' crystallisation space to form a co-crystal, if initial tests fail, through both traditional (e.g. solvent evaporation, solvent/anti-solvent diffusion) and modern (e.g. gel phase) experimental approaches. Current automation methods utilised to accelerate the discovery of co-crystals phases of organic molecules typically employ grams of analyte, and are often restricted to the production of crystalline powders, rather than single crystals suitable for SCXRD.

### BRIEF SUMMARY OF THE DISCLOSURE

In accordance with the present invention, there is provided a method of forming a co-crystal comprising a chemical molecule or a salt thereof, and a co-former, or a salt thereof, the method comprising:
a1) obtaining a first solution, said first solution comprising the chemical molecule or the salt thereof dissolved in an organic medium;
a2) obtaining a second solution, said second solution comprising the co-former or the salt thereof dissolved in an organic medium;
b) forming a droplet, the droplet comprising a first liquid and a second liquid; wherein the first liquid comprises a mixture of the first and second solutions; and the second liquid is an oil; and
c) allowing the co-crystal comprising the chemical molecule, or the salt thereof, and the co-former, or the salt thereof, to form in the droplet.

The inventors have found that co-crystals of chemicals (e.g. molecules) can be formed with a high enough quality for single crystal X-ray diffraction (SCXRD) even when only very small (e.g. <1 mg) amounts of the molecule are available. This is achieved using a droplet as described above. Without wishing to be bound by theory, it is believed that the oil slows down the evaporation of the organic medium allowing the concentration gradient of the molecule in the organic medium to change at a more controlled rate. Furthermore, the inventors have found that forming the molecule and the co-former(s) in separate solutions before forming the crystallisation droplet provides a better screen for co-crystals. The relative quantities of the compound and the co-former can be varied in a more controlled manner. Without wishing to be bound by theory, it is believed that the formation of the crystallisation droplet from two or more solutions allows for inhomogeneous distribution of the compound and the co-former within the droplet, which when coupled with the minimal convection within a small droplet, results in microenvironments within in the droplet containing varied concentrations and ratios. This results in improved access to co-crystal single crystals.

The method may be a method of forming a binary, ternary or quaternary co-crystal. Typically, where the first liquid comprises a mixture of the first and second solutions only, the method will be a method of forming a binary co-crystal.

The method may further comprise step a3): obtaining a third solution, said third solution comprising a second co-former or a salt thereof dissolved in an organic medium. In this embodiment, the first liquid comprises a mixture of the first, second and third solutions. In this embodiment, the co-former of the second solution may be described as ‘a first co-former’. Typically, in this embodiment, the co-crystal formed in the method comprises the chemical molecule and both co-formers (with each component being optionally present as a salt thereof). In this embodiment the method may therefore be a method of forming a ternary co-crystal.

In addition to step a3), the method may further comprise step a4): obtaining a fourth solution, said fourth solution comprising a third co-former or a salt thereof dissolved in an organic medium. In this embodiment, the first liquid comprises a mixture of the first, second, third and fourth solutions. Typically, in this embodiment, the co-crystal formed in the method comprises the chemical molecule and all three co-formers (with each component being optionally present as a salt thereof). In this embodiment the method may therefore be a method of forming a quaternary co-crystal.

In addition to step a4), the method may comprise a further step or steps of obtaining a further solution or solutions, said further solution or solutions comprising further co-formers or salts thereof dissolved in an organic medium. In these embodiments, the co-crystal formed in the method comprises the chemical molecule, the first, second and third co-formers, and the further co-former or co-formers (with each component being optionally present as a salt thereof).

The co-crystal formed maybe a solvate or hydrate.

The second solution may comprise a first co-former or a salt thereof and a second co-former or a salt thereof. In this embodiment, the co-crystal formed in the molecule comprises the chemical molecule and both co-formers (with each component being optionally present as a salt thereof).

The third solution may comprise a second co-former or a salt thereof and a third co-former or a salt thereof. In this embodiment, the co-crystal formed in the molecule comprises the chemical molecule and all three co-formers (with each component being optionally present as a salt thereof).

### First Solution

It may be that the first solution is a near saturated solution of the chemical molecule or a salt thereof.

The chemical molecule, or salt thereof, may be an organic molecule or salt thereof. The chemical molecule may be an organometallic molecule or salt thereof.

The chemical (e.g. organic) molecule or salt thereof may be a protein, a polypeptide, a polynucleotide, a polysaccharide or a salt thereof. The chemical (e.g. organic) molecule or salt thereof may be a conjugate of a small molecule with a species selected from a protein, a polypeptide, a polynucleotide, a polysaccharide or a salt thereof. The chemical (e.g. organic) molecule or salt thereof may be a protein.

Preferably, however, the chemical (e.g. organic) molecule will be a small molecule. For the purposes of this specification, a ‘small molecule’ may be considered to be a chemical (e.g. organic) molecule (or salt thereof) having a molecular mass below 5000 gmol⁻¹. It may be that the ‘small molecule’ has a molecular mass below 1000 gmol⁻¹. A ‘small molecule’ may be a chemical (e.g. organic) molecule (or salt thereof) comprising less than 100 atoms. A ‘small molecule’ may be a chemical (e.g. organic) molecule (or salt thereof) comprising less than 50 heavy atoms (heavy atoms being atoms other than hydrogen).

The chemical molecule may not be in the form of a salt. The chemical molecule may be a drug.

The first solution comprises the chemical (e.g. organic) molecule dissolved in an organic medium. The organic medium will typically comprise at least one organic solvent.

It may be that the organic component of the organic medium (e.g. the at least one organic solvent) is present in a total amount greater than 25% by volume of the organic medium. It may be that the organic component of the organic medium (e.g. the at least one organic solvent) is present in a total amount greater than 50% by volume of the organic medium. It may be that the organic component of the organic medium (e.g. the at least one organic solvent) is present in a total amount greater than 75% by volume of the organic medium. It may be that the organic component of the organic medium (e.g. the at least one organic solvent) is present in a total amount greater than 90% by volume of the organic medium. It may be that the organic component of the organic medium (e.g. the at least one organic solvent) is present in a total amount greater than 95% by volume of the organic medium. The organic medium typically comprises less than 20% water. The organic medium may comprise less than 5% water, e.g. less than 1% water. It may be that the organic medium does not comprise water. It may be that the organic medium comprises a single organic solvent.

Where the organic medium comprises a single organic solvent, it may be that the organic solvent is a polar organic solvent. It may be that the organic solvent is a polar protic organic solvent. It may be that the organic solvent is a polar aprotic organic solvent. Where the organic medium comprises more than one organic solvent, it may be that at least one organic solvent is a polar organic solvent. It may be that each organic solvent is a polar organic solvent. It may be that at least one organic solvent is a polar protic organic solvent. It may be that each organic solvent is a polar protic organic solvent. It may be that at least one organic solvent is a polar aprotic organic solvent. It may be that each organic solvent is a polar aprotic organic solvent.

Suitable organic solvents for use in the present invention are typically liquid at 25° C. Exemplary organic solvents include: hydrocarbons (e.g. pentane, hexane, heptane, octane, cyclohexane, petroleum ether); aromatic solvents (e.g. benzene, toluene, xylene, cumene, nitrobenzene); chlorinated solvents (e.g. chloroform, dichloromethane, 1,2-dichloroethane, chlorobenzene, 1,2-dichlorobenzene); fluorinated solvents (e.g. fluorobenzene, hexa-fluorobenzene); ethers (e.g. 1,4-dioxane, tetrahydrofuran, diethylether, diisopropylether, methyl-butyl-ether, dioxane, methyl or ethyl monoethers of propylene glycol or ethylene glycol, methyl or ethyl diethers of propylene glycol or ethylene glycol, methyl or ethyl monoethers of dipropylene glycol or diethylene glycol, methyl or ethyl diethers of dipropylene glycol or diethylene glycol); esters (e.g. ethyl acetate, isopropyl acetate), ketones (e.g. acetone, methylethylketone, dihydrolevoglucosenone); amides (e.g. N,N-dimethylformamide (DMF), N,N-dimethylacetamide (DMA), N-methyl-2-pyrrolidone (NMP)); sulfoxides (e.g. dimethylsulfoxide (DMSO)); alcohols (e.g. methanol, ethanol, i-propanol, n-propanol, n-butanol, t-butanol); diols (e.g. ethylene glycol, propylene glycol, 2-methyl-2,4-pentanediol); ureas (e.g. N,N′-dimethylpropyleneurea, 1,3-dimethyl-2-imidazolidinone); phosphoramides (e.g. hexamethylphosphoramide); and nitro compounds (e.g. nitromethane).

Suitable organic solvents include polar non-protic solvents, e.g. DMSO, DMF, DMA and NMP.

The at least one organic solvent may be a solvent selected from DMF, MeOH, 1,4-dioxane and MeNO₂.

Where the organic medium comprises a single organic solvent, it may be that the organic solvent has a boiling point greater than 50° C. It may be that the organic solvent has a boiling point greater than 80° C. It may be that the organic solvent has a boiling point greater than 125° C. It may be that the organic solvent has a boiling point in the range from 50° C. to 80° C. Where the organic medium comprises more than one organic solvent, it may be that at least one organic solvent has a boiling point greater than 50° C. It may be that at least one organic solvent has a boiling point greater than 80° C. It may be that at least one organic solvent has a boiling point greater than 125° C. It may be that at least one organic solvent has a boiling point in the range from 50° C. to 80° C. It may be that each organic solvent has a boiling point greater than 50° C. It may be that each organic solvent has a boiling point greater than 80° C. It may be that each organic solvent has a boiling point greater than 125° C. It may be that each organic solvent has a boiling point in the range from 50° C. to 80° C. It may be that the organic medium does not comprise a solvent with a boiling point greater than 250° C. It may be that the organic medium does not comprise a solvent with a boiling point greater than 200° C. It may be that the organic medium does not comprise a solvent with a boiling point greater than 150° C. The inventors have found that solvents with higher boiling points are particularly effective in the methods of the invention. Known crystallisation methods based on larger solvent volumes and sample size are generally unsuccessful or at best impractical with higher boiling point solvents. The rate of evaporation of even a high boiling point solvent is fast enough to provide a practical methodology where the volume of that high boiling point solvent is low. For the absence of doubt, the boiling points referred to above are the boiling points at 1 atm.

The organic medium will typically be selected such that the chemical (e.g. organic) molecule, or salt thereof, is soluble in the organic medium. The organic medium will typically comprise at least one solvent in which the chemical (e.g. organic) molecule, or salt thereof, is soluble.

### Second Solution

The second solution comprises the co-former (e.g. first co-former) or salt thereof dissolved in an organic medium. The organic medium will typically comprise at least one organic solvent.

It may be that the co-former (e.g. first co-former) is not a drug. Alternatively, the co-former (e.g. first co-former) may be a drug.

It may be that the second solution is a near saturated solution of the co-former (e.g. first co-former) or a salt thereof.

The co-former (e.g. first co-former) may not be in the form of a salt.

It may be that the organic component of the organic medium (e.g. the at least one organic solvent) is present in a total amount greater than 25% by volume of the organic medium. It may be that the organic component of the organic medium (e.g. the at least one organic solvent) is present in a total amount greater than 50% by volume of the organic medium. It may be that the organic component of the organic medium (e.g. the at least one organic solvent) is present in a total amount greater than 75% by volume of the organic medium. It may be that the organic component of the organic medium (e.g. the at least one organic solvent) is present in a total amount greater than 90% by volume of the organic medium. It may be that the organic component of the organic medium (e.g. the at least one organic solvent) is present in a total amount greater than 95% by volume of the organic medium. The organic medium typically comprises less than 20% water. The organic medium may comprise less than 5% water, e.g. less than 1% water. It may be that the organic medium does not comprise water, i.e. the organic medium comprises a single organic solvent.

Where the organic medium comprises a single organic solvent, it may be that the organic solvent is a polar organic solvent. It may be that the organic solvent is a polar protic organic solvent. It may be that the organic solvent is a polar aprotic organic solvent. Where the organic medium comprises more than one organic solvent, it may be that at least one organic solvent is a polar organic solvent. It may be that each organic solvent is a polar organic solvent. It may be that at least one organic solvent is a polar protic organic solvent. It may be that each organic solvent is a polar protic organic solvent. It may be that at least one organic solvent is a polar aprotic organic solvent. It may be that each organic solvent is a polar aprotic organic solvent.

Suitable organic solvents for use in the present invention are typically liquid at 25° C. Exemplary organic solvents include: hydrocarbons (e.g. pentane, hexane, heptane, octane, cyclohexane, petroleum ether); aromatic solvents (e.g. benzene, toluene, xylene, cumene, nitrobenzene); chlorinated solvents (e.g. chloroform, dichloromethane, 1,2-dichloroethane, chlorobenzene, 1,2-dichlorobenzene); fluorinated solvents (e.g. fluorobenzene, hexa-fluorobenzene); ethers (e.g. 1,4-dioxane, tetrahydrofuran, diethylether, diisopropylether, methyl-butyl-ether, dioxane, methyl or ethyl monoethers of propylene glycol or ethylene glycol, methyl or ethyl diethers of propylene glycol or ethylene glycol, methyl or ethyl monoethers of dipropylene glycol or diethylene glycol, methyl or ethyl diethers of dipropylene glycol or diethylene glycol); esters (e.g. ethyl acetate, isopropyl acetate), ketones (e.g. acetone, methylethylketone, dihydrolevoglucosenone); amides (e.g. N,N-dimethylformamide (DMF), N,N-dimethylacetamide (DMA), N-methyl-2-pyrrolidone (NMP)); sulfoxides (e.g. dimethylsulfoxide (DMSO)); alcohols (e.g. methanol, ethanol, i-propanol, n-propanol, n-butanol, t-butanol); diols (e.g. ethylene glycol, propylene glycol, 2-methyl-2,4-pentanediol); ureas (e.g. N,N′-dimethylpropyleneurea, 1,3-dimethyl-2-imidazolidinone); phosphoramides (e.g. hexamethylphosphoramide); and nitro compounds (e.g. nitromethane).

Suitable organic solvents include polar non-protic solvents, e.g. DMSO, DMF, DMA and NMP.

The at least one organic solvent may be a solvent selected from DMF, MeOH, 1,4-dioxane and MeNO₂.

Where the organic medium comprises a single organic solvent, it may be that the organic solvent has a boiling point greater than 50° C. It may be that the organic solvent has a boiling point greater than 80° C. It may be that the organic solvent has a boiling point greater than 125° C. It may be that the organic solvent has a boiling point in the range from 50° C. to 80° C. Where the organic medium comprises more than one organic solvent, it may be that at least one organic solvent has a boiling point greater than 50° C. It may be that at least one organic solvent has a boiling point greater than 80° C. It may be that at least one organic solvent has a boiling point greater than 125° C. It may be that at least one organic solvent has a boiling point in the range from 50° C. to 80° C. It may be that each organic solvent has a boiling point greater than 50° C. It may be that each organic solvent has a boiling point greater than 80° C. It may be that each organic solvent has a boiling point greater than 125° C. It may be that each organic solvent has a boiling point in the range from 50° C. to 80° C. It may be that the organic medium does not comprise a solvent with a boiling point greater than 250° C. It may be that the organic medium does not comprise a solvent with a boiling point greater than 200° C. It may be that the organic medium does not comprise a solvent with a boiling point greater than 150° C.

The organic medium will typically be selected such that the co-former (e.g. first co-former), or salt thereof, is soluble in the organic medium. The organic medium will typically comprise at least one solvent in which the co-former (e.g. first co-former), or salt thereof, is soluble.

### Third Solution

The (optional) third solution comprises the second co-former or salt thereof dissolved in an organic medium. The organic medium will typically comprise at least one organic solvent.

It may be that the third solution is a near saturated solution of the second co-former or a salt thereof.

The second co-former may not be in the form of a salt. It may be that the second co-former is a drug. It may be that the second co-former is not a drug.

It may be that the organic component of the organic medium (e.g. the at least one organic solvent) is present in a total amount greater than 25% by volume of the organic medium. It may be that the organic component of the organic medium (e.g. the at least one organic solvent) is present in a total amount greater than 50% by volume of the organic medium. It may be that the organic component of the organic medium (e.g. the at least one organic solvent) is present in a total amount greater than 75% by volume of the organic medium. It may be that the organic component of the organic medium (e.g. the at least one organic solvent) is present in a total amount greater than 90% by volume of the organic medium. It may be that the organic component of the organic medium (e.g. the at least one organic solvent) is present in a total amount greater than 95% by volume of the organic medium. The organic medium typically comprises less than 20% water. The organic medium may comprise less than 5% water, e.g. less than 1% water. It may be that the organic medium does not comprise water, i.e. the organic medium comprises a single organic solvent.

Where the organic medium comprises a single organic solvent, it may be that the organic solvent is a polar organic solvent. It may be that the organic solvent is a polar protic organic solvent. It may be that the organic solvent is a polar aprotic organic solvent. Where the organic medium comprises more than one organic solvent, it may be that at least one organic solvent is a polar organic solvent. It may be that each organic solvent is a polar organic solvent. It may be that at least one organic solvent is a polar protic organic solvent. It may be that each organic solvent is a polar protic organic solvent. It may be that at least one organic solvent is a polar aprotic organic solvent. It may be that each organic solvent is a polar aprotic organic solvent.

Suitable organic solvents for use in the present invention are typically liquid at 25° C. Exemplary organic solvents include: hydrocarbons (e.g. pentane, hexane, heptane, octane, cyclohexane, petroleum ether); aromatic solvents (e.g. benzene, toluene, xylene, cumene, nitrobenzene); chlorinated solvents (e.g. chloroform, dichloromethane, 1,2-dichloroethane, chlorobenzene, 1,2-dichlorobenzene); fluorinated solvents (e.g. fluorobenzene, hexa-fluorobenzene); ethers (e.g. tetrahydrofuran, diethylether, diisopropylether, methyl-butyl-ether, dioxane, methyl or ethyl monoethers of propylene glycol or ethylene glycol, methyl or ethyl diethers of propylene glycol or ethylene glycol, methyl or ethyl monoethers of dipropylene glycol or diethylene glycol, methyl or ethyl diethers of dipropylene glycol or diethylene glycol); esters (e.g. 1,4-dioxane, ethyl acetate, isopropyl acetate), ketones (e.g. acetone, methylethylketone, dihydrolevoglucosenone); amides (e.g. N,N-dimethylformamide (DMF), N,N-dimethylacetamide (DMA), N-methyl-2-pyrrolidone (NMP)); sulfoxides (e.g. dimethylsulfoxide (DMSO)); alcohols (e.g. methanol, ethanol, i-propanol, n-propanol, n-butanol, t-butanol); diols (e.g. ethylene glycol, propylene glycol, 2-methyl-2,4-pentanediol); ureas (e.g. N,N′-dimethylpropyleneurea, 1,3-dimethyl-2-imidazolidinone); phosphoramides (e.g. hexamethylphosphoramide); and nitro compounds (e.g. nitromethane).

Suitable organic solvents include polar non-protic solvents, e.g. DMSO, DMF, DMA and NMP.

The at least one organic solvent may be a solvent selected from DMF, MeOH, 1,4-dioxane and MeNO₂.

Where the organic medium comprises a single organic solvent, it may be that the organic solvent has a boiling point greater than 50° C. It may be that the organic solvent has a boiling point greater than 80° C. It may be that the organic solvent has a boiling point greater than 125° C. It may be that the organic solvent has a boiling point in the range from 50° C. to 80° C. Where the organic medium comprises more than one organic solvent, it may be that at least one organic solvent has a boiling point greater than 50° C. It may be that at least one organic solvent has a boiling point greater than 80° C. It may be that at least one organic solvent has a boiling point greater than 125° C. It may be that at least one organic solvent has a boiling point in the range from 50° C. to 80° C. It may be that each organic solvent has a boiling point greater than 50° C. It may be that each organic solvent has a boiling point greater than 80° C. It may be that each organic solvent has a boiling point greater than 125° C. It may be that each organic solvent has a boiling point in the range from 50° C. to 80° C. It may be that the organic medium does not comprise a solvent with a boiling point greater than 250° C. It may be that the organic medium does not comprise a solvent with a boiling point greater than 200° C. It may be that the organic medium does not comprise a solvent with a boiling point greater than 150° C.

The organic medium will typically be selected such that the second co-former, or salt thereof, is soluble in the organic medium. The organic medium will typically comprise at least one solvent in which the second co-former, or salt thereof, is soluble.

### Fourth and Further Solutions

The (optional) fourth, and any further, solutions may be as defined above for the second and third solutions.

### First Liquid

It may be that none of the chemical molecule and the co-former(s) are in the form of a salt.

The methods of the invention are particularly effective in the crystallisation of very small amounts of material. Thus, the first liquid may comprise less than 1 mg of the chemical (e.g. organic) molecule, or salt thereof. The first liquid may comprise less than 500 μg of the chemical (e.g. organic) molecule, or salt thereof. The first liquid may comprise less than 100 μg of the chemical (e.g. organic) molecule, or salt thereof. The first liquid may comprise less than 10 μg of the chemical (e.g. organic) molecule, or salt thereof. The first liquid may comprise less than 5 μg of the chemical (e.g. organic) molecule, or salt thereof.

The total volume of the first liquid in the droplet may be less than 1 μL. The total volume of the first liquid in the droplet may be less than 500 nL. The total volume of the first liquid in the droplet may be less than 200 nL. The total volume of the first liquid in the droplet may be less than 100 nL. Without wishing to be bound by theory, it is believed that the small size of the sample of the first liquid contributes to the success of the method in providing crystals suitable for SCXRD. This may be because the effective lack of convection currents within the sample of the first liquid allows the steady growth of co-crystals that start to form and/or the minimal nucleation points present in the sample of the first liquid allows initiation of the formation of minimum number of crystals.

The concentration of the chemical (e.g. organic) molecule, or salt thereof, within the first liquid may be in the range from 1 mg/mL to 1 g/mL. The concentration of the chemical (e.g. organic) molecule, or salt thereof, within the first liquid may be in the range from 10 mg/mL to 500 mg/mL. The concentration of the chemical (e.g. organic) molecule, or salt thereof, within the first liquid may be in the range from 10 mg/mL to 800 mg/mL.

The concentration of the chemical (e.g. organic) molecule, or salt thereof, within the first liquid may be in the range from 1 mg/mL to 200 mg/mL. The concentration of the chemical (e.g. organic) molecule, or salt thereof, within the first liquid may be in the range from 5 mg/mL to 150 mg/mL.

The concentration of the co-former, or salt thereof, within the first liquid may be in the range from 1 mg/mL to 1 g/mL. The concentration of the co-former, or salt thereof, within the first liquid may be in the range from 10 mg/mL to 500 mg/mL. The concentration of the co-former, or salt thereof, within the first liquid may be in the range from 10 mg/mL to 800 mg/mL.

The concentration of the co-former (e.g. the first co-former), or salt thereof, within the first liquid may be in the range from 1 mg/mL to 200 mg/mL. The concentration of the co-former, or salt thereof, within the first liquid may be in the range from 5 mg/mL to 150 mg/mL.

It may be that the organic medium in the first solution and the second solution are the same. It may be that the organic medium in the first solution and the second solution are different. Thus, it may be that the first solution comprises the chemical molecule or salt thereof dissolved in a first organic medium; and the second solution comprises a co-former or a salt thereof dissolved in a second organic medium.

Where a third solution is also used, the organic medium in the third solution may be the same as in the first solution. The organic medium in the third solution may be the same as in the second solution. The organic medium in the third solution may be the same as in the first and second solutions. The organic medium in the third solution may be different to those in the first and second solutions.

It may be that the organic medium of the first solution is the same as the organic medium of the second solution. It may be that the organic medium of the first solution is different to the organic medium of the second solution. It may be that the organic component of the organic medium of the first solution is different to the organic component of the organic medium of the second solution. It may be that the organic medium of the first solution comprises an organic solvent and the organic medium of the second solution comprises an organic solvent, wherein the organic solvent of the first solution is different to the organic solvent of the second solution.

In an embodiment, the droplet comprises a third solution comprising a second co-former or a salt thereof dissolved in an organic medium. It may be that the organic medium of the first solution is the same as the organic medium of the third solution. It may be that the organic medium of the first solution is different to the organic medium of the third solution. It may be that the organic component of the organic medium of the first solution is different to the organic component of the organic medium of the third solution. It may be that the organic medium of the first solution comprises an organic solvent and the organic medium of the third solution comprises an organic solvent, wherein the organic solvent of the first solution is different to the organic solvent of the third solution. It may be that the organic medium of the second solution is the same as the organic medium of the third solution. It may be that the organic medium of the second solution is different to the organic medium of the third solution. It may be that the organic component of the organic medium of the second solution is different to the organic component of the organic medium of the third solution. It may be that the organic medium of the second solution comprises an organic solvent and the organic medium of the third solution comprises an organic solvent, wherein the organic solvent of the second solution is different to the organic solvent of the third solution.

In embodiments where the first liquid comprises a mixture of the first, second and third solutions, it may be that the concentrations of the chemical (e.g. organic) molecule, or salt thereof, the first co-former, or salt thereof, and the second co-former, or salt thereof, within the first liquid are each independently in the range from 1 mg/mL to 150 mg/mL. The concentrations of the chemical (e.g. organic) molecule, or salt thereof, the first co-former, or salt thereof, and the second co-former, or salt thereof, within the first liquid may be each independently in the range from 1 mg/mL to 100 mg/mL.

In embodiments where the first liquid comprises a mixture of the first, second, third and fourth solutions, it may be that the concentrations of the chemical (e.g. organic) molecule, or salt thereof, the first co-former, or salt thereof, the second co-former, or salt thereof and the third co-former, or salt thereof, within the first liquid are each independently in the range from 1 mg/mL to 100 mg/mL. The concentrations of the chemical (e.g. organic) molecule, or salt thereof, the first co-former, or salt thereof, the second co-former, or salt thereof, and the third co-former, or salt thereof, within the first liquid may be each independently in the range from 1 mg/mL to 50 mg/mL.

### Second Liquid

The second liquid is an oil. The oil may be fully miscible with the first liquid. More typically, however, is that the oil is partially miscible or immiscible with the first liquid.

The oil will typically be selected such that the chemical (e.g. organic) molecule, or salt thereof, is substantially insoluble in the oil. It may be that the chemical (e.g. organic) molecule, or salt thereof, is partially soluble in the oil.

The oil will typically have a viscosity (at 25° C. and 1 atm) in the range from 0.1 cP to 1200 cP. The oil may have a viscosity (at 25° C. and 1 atm) in the range 1 cP to 500 cP. The oil may have a viscosity (at 25° C. and 1 atm) in the range 2 cP to 60 cP.

The oil may be a mineral oil, a paraffin oil, a silicon oil or a perfluorinated oil. The oil may be a silicon oil, e.g. polydimethylsiloxane. The oil may be a paraffin oil. The oil may be a perfluorinated oil.

The oil may be a perfluorocarbon, e.g. perfluorooctane.

The oil may be a perfluorinated amine. The oil may be a perfluorinated tertiary amine. The oil may be a perfluorinated tertiary amine with an average molecular weight in the range 200 to 900 gmol⁻¹. The oil may be a perfluorinated tertiary amine with an average molecular weight in the range 300 to 700 gmol⁻¹. The oil may be an oil selected from perfluorotripentylamine, perfluorotributylamine, perfluorodibutylmethylamine, or a mixture thereof. Illustrative examples include: Fluorinert™ FC-40 (CAS Number 51142-49-5), Fluorinert™ FC-43 (CAS Number 311-89-7), Fluorinert™ FC-770 (CAS Number 1093615-61-2 Fluorinert™ FC-104 (CAS Number 70852-06-1).

The oil may be a perfluoroether or perfluoropolyether. Illustrative examples include: perfluoro(2-n-butyltetrahydrofuran), Fomblin YR-1800, Fomblin Y, Fomblin-M, Fomblin Z-15.

It may be that the oil is not paraffin oil.

The total volume of the second liquid in the droplet may be less than 1 μL. The total volume of the second liquid in the droplet may be less than 500 nL. The total volume of the second liquid in the droplet may be less than 350 nL. The total volume of the second liquid in the droplet may be less than 250 nL.

### Methods

Step a1) may comprise forming the first solution by dissolving the chemical molecule or a salt thereof in the organic medium.

The concentration of the chemical (e.g. organic) molecule, or salt thereof, within the organic medium may be in the range from 1 mg/mL to 1 g/mL. The concentration of the chemical (e.g. organic) molecule, or salt thereof, within the organic medium may be in the range from 10 mg/mL to 500 mg/mL. The concentration of the chemical (e.g. organic) molecule, or salt thereof, within the organic medium may be in the range from 10 mg/mL to 800 mg/mL.

The concentration of the chemical molecule or salt thereof within the organic medium may be from 5 mg/mL to 250 mg/mL. The concentration of the chemical molecule or salt thereof in within the organic medium may be from 10 mg/mL to 200 mg/mL.

Step a2) may comprise forming the second solution by dissolving the co-former (e.g. first co-former) or a salt thereof in the organic medium.

The concentration of the co-former (e.g. first co-former) or salt thereof, within the organic medium may be in the range from 1 mg/mL to 1 g/mL. The concentration of the co-former (e.g. first co-former), or salt thereof, within the organic medium may be in the range from 10 mg/mL to 500 mg/mL. The concentration of the co-former (e.g. first co-former), or salt thereof, within the organic medium may be in the range from 10 mg/mL to 800 mg/mL.

The concentration of the co-former (e.g. first co-former) or salt thereof, within the organic medium may be in the range from 5 mg/mL to 250 mg/mL. The concentration of the co-former (e.g. first co-former) or salt thereof, within the organic medium may be in the range from 10 mg/mL to 200 mg/mL.

Step a3) may comprise forming the third solution by dissolving the second co-former or a salt thereof in the organic medium.

The concentration of the second co-former or salt thereof within the organic medium may be in the range from 1 mg/mL to 1 g/mL. The concentration of the second co-former, or salt thereof, within the organic medium may be in the range from 10 mg/mL to 500 mg/mL. The concentration of the second co-former, or salt thereof, within the organic medium may be in the range from 10 mg/mL to 800 mg/mL.

The concentration of the second co-former or salt thereof, within the organic medium may be in the range from 5 mg/mL to 250 mg/mL. The concentration of the second co-former or salt thereof, within the organic medium may be in the range from 10 mg/mL to 200 mg/mL.

Step a4) may comprise forming the fourth solution by dissolving the third co-former or a salt thereof in the organic medium.

The concentration of the third co-former or salt thereof within the organic medium may be in the range from 1 mg/mL to 1 g/mL. The concentration of the third co-former, or salt thereof, within the organic medium may be in the range from 10 mg/mL to 800 mg/mL. The concentration of the third co-former, or salt thereof, within the organic medium may be in the range from 10 mg/mL to 500 mg/mL.

The concentration of the third co-former or salt thereof, within the organic medium may be in the range from 5 mg/mL to 250 mg/mL. The concentration of the third co-former or salt thereof, within the organic medium may be in the range from 10 mg/mL to 200 mg/mL.

The co-crystal may comprise only the chemical (e.g. organic) molecule or salt thereof and the co-former(s) or salt(s) thereof. It may be that the co-crystal formed is a solvate of the chemical (e.g. organic) molecule or salt thereof comprising the chemical (e.g. organic) molecule (or salt thereof), the co-former(s) or salt(s) thereof and a solvent that is comprised in the organic medium.

It may be that the second liquid forms a coating over the first liquid. The second liquid may encapsulate the first liquid. Alternatively, it may be that a small portion of the surface of the first liquid is not coated with the second liquid. Thus, it may be that greater than 90%, e.g. 95%, of the surface of the first liquid (i.e. the surface of the first liquid that is not in contact with the support surface) is coated with the second liquid.

The ratio first liquid:second liquid may be in the range from 2:1 to 1:20 by volume. The ratio first liquid:second liquid may be in the range from 1:1 to 1:10 by volume. The ratio first liquid:second liquid may be in the range from 1:2 to 1:6 by volume.

Typically, once the droplet comprising the first and second liquid is formed, the droplet (including the first liquid and the second liquid) is not moved while the crystal is forming. It may be that the first liquid does not move. It may be that the second liquid does not move. Thus, the first liquid is not in contact with a continuous flow of the second liquid. Therefore, it may be that the droplet comprising the first and second liquid is not exposed to a continuous flow of an external liquid.

The droplet may be a sitting droplet. Thus, it may be that the sitting droplet comprising the first and the second liquid does not move. Alternatively, the droplet may be a hanging droplet. Thus, it may be that the hanging droplet comprising the first and the second liquid does not move. The amount, surface tension and the viscosity of the oil will be chosen according to the form of the droplet.

A seed crystal may be present in the droplet. The seed crystal may comprise the co-crystal formed by the method of the present invention, i.e. a co-crystal comprising the chemical molecule, or the salt thereof, and the co-former, or the salt thereof. The seed crystal may comprise a co-crystal comprising the chemical molecule. The seed crystal may comprise a co-crystal comprising the first co-former. Where the first liquid comprises a third solution, the seed crystal may comprise a co-crystal comprising the second co-former. The seed crystal may comprise a crystal of another chemical molecule that is different to the chemical molecule and the co-former (or co-formers, if present). Typically, however, no seed crystal is present.

The step c) may be conducted in the presence of an antisolvent. The antisolvent may be within the droplet or the antisolvent may be not part of the droplet, e.g. the antisolvent may be arranged relative to the droplet such that vapour from the antisolvent can come into contact with the droplet. The antisolvent may be in a second droplet (e.g. not part of the first droplet formed in step b)) or, more typically, the antisolvent will be in an open vessel.

The antisolvent may be comprised in the droplet. Where this is the case, the antisolvent may form part of the first liquid. It may be that the antisolvent does not form part of the first liquid. Alternatively, the antisolvent may initially (i.e. when the droplet is formed) form a third liquid. It may be that the third liquid coats the first liquid and the second liquid coats the third liquid. It may be that the first liquid coats the third liquid and the second liquid coats the first liquid. It may be that the first liquid and the third liquid are in contact and the second liquid coats the surfaces of the third liquid and the first liquid that are not in contact with each other or the support surface on which the droplet has been formed.

It may be that the first liquid and the third liquid are immiscible. It may be that the first liquid and the third liquid are slightly miscible and mix during the course of step c).

The antisolvent may be a solvent selected from the list of suitable organic solvents referred to above. The antisolvent may be water. It may be that the antisolvent is not water. The antisolvent may be ethanol. The antisolvent may be a non-polar solvent, e.g. toluene.

Typically, the antisolvent will be selected such that the chemical molecule is less soluble in the antisolvent than it is in the first liquid.

It may be that the droplet is formed in a vessel, e.g. a well. This might typically be the case where the droplet is a sitting drop. It may be that the method comprises a further step, once the droplet is formed, of sealing the vessel, e.g. well, in which the droplet is formed. It may be that the droplet is formed on a surface. This might typically be the case where the droplet is a hanging drop. It may be that the method comprises a further step, once the droplet is formed, of sealing a vessel, e.g. well, with the surface on which the droplet is formed. Use of a sealed vessel, e.g. a sealed well, can slow the evaporation of the organic medium and can provide better quality crystals. The inventors have, however, formed good quality crystals using the methods of the invention in which the droplet was not in a sealed vessel.

Where an antisolvent is used and that antisolvent is not part of the droplet, the droplet will typically be placed in a sealed system.

The droplet will be formed on a support surface.

The support surface on which the droplet is formed may be flat. The support surface on which the droplet is formed may be concave. This embodiment applies particularly to sitting droplets. The support surface on which the droplet is formed may be convex. This applies particularly to hanging droplets.

The support surface on which the droplet is formed may be glass. The support surface on which the droplet is formed may be plastic.

The support surface on which the droplet is formed may be unmodified. Alternatively, it may be modified to alter its affinity with the first liquid and/or the second liquid, e.g. it may be coated with a material that alters its properties.

In preferred embodiments, the first, second and, optionally, the third and any further solutions, mix to form the first liquid after the droplet is formed (e.g. after the first, second and, optionally, the third and any further solutions, are added to the second liquid to form the droplet). Here the droplet may be formed from the first, second and, optionally, the third and any further solutions, wherein the first, second and, optionally, the third and any further solutions are separate solutions, or are unmixed, or partially mixed.

The droplet is typically formed by first forming a droplet of the second liquid and then injecting the solutions of the first liquid (i.e. the first, second and, optionally, the third and any further solutions) into the droplet of the second liquid to form the droplet.

It may be that the droplet is formed by first forming a droplet of the second liquid and then injecting the first liquid (i.e. comprising a mixture of the first, second and, optionally, the third and any further solutions) into the droplet of the second liquid to form the droplet. Alternatively, the droplet may be formed by first forming a droplet of the second liquid and injecting the first, second and, optionally, the third and any further solutions into the droplet, wherein the solutions are unmixed or partially mixed. Once in the droplet, the solutions will mix to form the first liquid.

Alternatively, it may be that a droplet or droplets of the solutions of the first liquid (i.e. the first, second and, optionally, the third and any further solutions) is formed and the second liquid is coated onto the droplet or droplets of the solutions of the first liquid to form the droplet.

It may be that a droplet of the first liquid is formed and the second liquid is coated onto the droplet of the first liquid to form the droplet. Alternatively, it may be that a droplet comprising the solutions of the first liquid is formed, wherein the solutions are unmixed or partially mixed within the droplet (unmixed or partially mixed solutions in the same droplet will share a liquid interface). The second liquid is then coated onto the droplet of the solutions of the first liquid to form the droplet. The solutions will mix in the droplet to form the first liquid.

Alternatively, it may be that a separate droplet of each solution of the first liquid is formed. The second liquid is then coated onto and over the droplets of the solutions of the first liquid to form the droplet. The solution droplets will mix in the droplet to form the first liquid.

The support surface on which the droplet is formed may be the bottom of a vessel. This is typically the case with a sitting droplet. The vessel may be a well, e.g. a well of a multi-well plate.

It may be that the droplet is formed on the support surface and that support surface is then inverted such that the droplet is below the support surface. This is known as a hanging droplet. Alternatively, it may be that a droplet of the second liquid is formed on the support surface, the surface is inverted to form a hanging droplet of the second liquid and then the first liquid is injected into the second liquid.

The step of forming the droplet involves mixing the first solution and the second solution (and optionally the third solution and any further solution(s)) to form the first liquid.

It may be that the first solution and the second solution (and optionally the third solution and any further solution(s)) are mixed, or partially mixed, before contact with the second liquid.

It may be that the droplet is formed by: forming a droplet of the second liquid; taking the first solution and the second solution (and optionally the third and any further solution(s)) up into a syringe; and injecting the first solution and the second solution (and optionally the third and any further solution(s)) into the second liquid to form the droplet.

It may be that the droplet is formed by: forming a droplet of the second liquid; taking the first solution and the second solution (and optionally the third and any further solution(s)) up into a syringe to form the first liquid in the syringe; and injecting the first liquid into the second liquid to form the droplet.

It may be that the droplet is formed by: forming a droplet of the second liquid; taking the first solution and the second solution (and optionally the third and any further solution(s)) up into a syringe so that first solution and the second solution (and optionally the third and any further solution(s)) share a liquid interface and/or are partially mixed; and injecting the first solution and the second solution (and optionally the third and any further solution(s)) into the second liquid to form the droplet.

It may be that the droplet is formed by: taking the first solution and the second solution (and optionally the third solution) up into a syringe to form the first liquid in the syringe; forming a droplet of the first liquid; and coating the second liquid onto the droplet of the first liquid to form the droplet.

It may be that the droplet is formed by: taking the first solution and the second solution (and optionally the third solution) up into separate syringes; mixing the solutions to form a droplet of the first liquid; and coating the second liquid onto the droplet of the first liquid to form the droplet.

It may be that the first solution and the second solution (and optionally the third solution) are mixed after contact with the second liquid.

It may be that the droplet is formed by: forming a droplet of the second liquid; taking the first solution and the second solution (and optionally the third solution) up into separate syringes; and injecting the first solution and the second solution (and optionally the third solution and any further solutions) separately into the second liquid to form the droplet. It may be that the first and second solutions (and optionally the third solution and any further solutions) are injected separately into the second liquid so that they are initially spaced apart within the droplet, i.e. so that the first and second solutions are initially separated within the droplet by a portion of the second liquid.

It may be that the droplet is formed by: forming a droplet of the second liquid; taking the first solution, a portion, or portions, of the second liquid, and the second solution (and, optionally, the third solution and any further solutions) up into a syringe so that each solution is partitioned from the next by a portion of the second liquid; and injecting the first solution, the portion or portions of the second liquid, and the second solution (and optionally the third solution and any further solution(s)) into the second liquid to form the droplet.

In any of the above embodiments in which the first solution and the second solution (and optionally the third and any further solution(s)) are taken up into a syringe, it may be that said solutions are injected into the second liquid, or injected to from a droplet, within 10 minutes or less, e.g. 5 minutes or less, from being taken up into the syringe. It may be that said solutions are injected into the second liquid, or injected to from a droplet, within 2 minutes or less, e.g. 1 minute or less, from being taken up into the syringe. Preferably, said solutions are injected into the second liquid, or injected to from a droplet, within 30 seconds or less, e.g. 10 seconds or less, from being taken up into the syringe.

It may be that the first solution and the second solution (and optionally the third and any further solution(s)) are taken up into a syringe at an aspiration flow rate of less than 5 mm min⁻¹, e.g. less than 4 mm min⁻¹. It may be that the first solution and the second solution (and optionally the third and any further solution(s)) are taken up into a syringe at an aspiration flow rate of less than 3 mm min⁻¹, e.g. less than 2 mm min⁻¹.

It may be that the volume ratio of first solution:second solution is in the range from 1:25 to 25:1, e.g. from 1:10 to 10:1. It may be that the volume ratio of first solution:second solution is in the range from 1:4 to 4:1. It may be that the volume ratio of first solution:second solution is in the range from 1:3 to 3:1.

It may be that the volume ratio of first solution:third solution is in the range from 1:25 to 25:1, e.g. from 1:10 to 10:1. It may be that the volume ratio of first solution:third solution is in the range from 1:4 to 4:1. It may be that the volume ratio of first solution:third solution is in the range from 1:3 to 3:1. It may be that the volume ratio of second solution:third solution is in the range from 1:25 to 25:1, e.g. from 1:10 to 10:1. It may be that the volume ratio of second solution:third solution is in the range from 1:4 to 4:1. It may be that the volume ratio of second solution:third solution is in the range from 1:3 to 3:1.

It may be that the volume ratio of first solution:fourth solution is in the range from 1:25 to 25:1, e.g. from 1:10 to 10:1. It may be that the volume ratio of first solution:fourth solution is in the range from 1:4 to 4:1. It may be that the volume ratio of first solution:fourth solution is in the range from 1:3 to 3:1. It may be that the volume ratio of second solution:fourth solution is in the range from 1:25 to 25:1, e.g. from 1:10 to 10:1. It may be that the volume ratio of second solution:fourth solution is in the range from 1:4 to 4:1. It may be that the volume ratio of second solution:fourth solution is in the range from 1:3 to 3:1. It may be that the volume ratio of third solution:fourth solution is in the range from 1:25 to 25:1, e.g. from 1:10 to 10:1. It may be that the volume ratio of third solution:fourth solution is in the range from 1:4 to 4:1. It may be that the volume ratio of third solution:fourth solution is in the range from 1:3 to 3:1.

It may be that the stoichiometric ratio of the chemical molecule in the first solution:co-former in the second solution is from 10:1 to 1:10, e.g. from 5:1 to 1:5. It may be that the stoichiometric ratio of the chemical molecule in the first solution:co-former in the second solution is from 10:1 to 1:10, e.g. from 5:1 to 1:5. It may be that the stoichiometric ratio of the chemical molecule in the first solution:co-former in the second solution is from 2:1 to 1:2. It may be that the stoichiometric ratio of the chemical molecule in the first solution:co-former in the second solution is around 1:1.

It may be that the stoichiometric ratio of the chemical molecule in the first solution:co-former in the third solution is from 10:1 to 1:10, e.g. from 5:1 to 1:5. It may be that the stoichiometric ratio of the chemical molecule in the first solution:co-former in the third solution is from 10:1 to 1:10, e.g. from 5:1 to 1:5. It may be that the stoichiometric ratio of the chemical molecule in the first solution:co-former in the third solution is from 2:1 to 1:2. It may be that the stoichiometric ratio of the chemical molecule in the first solution:co-former in the third solution is around 1:1.

It may be that the stoichiometric ratio of the chemical molecule in the first solution:co-former in the fourth solution is from 10:1 to 1:10, e.g. from 5:1 to 1:5. It may be that the stoichiometric ratio of the chemical molecule in the first solution:co-former in the fourth solution is from 10:1 to 1:10, e.g. from 5:1 to 1:5. It may be that the stoichiometric ratio of the chemical molecule in the first solution:co-former in the fourth solution is from 2:1 to 1:2. It may be that the stoichiometric ratio of the chemical molecule in the first solution:co-former in the fourth solution is around 1:1.

It may be that the concentration (mg/mL) ratio of the chemical molecule in the first solution:co-former in the second solution is from 15:1 to 1:15. It may be that the concentration ratio of the chemical molecule in the first solution:co-former in the second solution is from 12:1 to 1:12. It may be that the concentration ratio of the chemical molecule in the first solution:co-former in the second solution is from 10:1 to 1:10.

It may be that the concentration (mg/mL) ratio of the chemical molecule in the first solution:co-former in the third solution is from 15:1 to 1:15. It may be that the concentration ratio of the chemical molecule in the first solution:co-former in the third solution is from 12:1 to 1:12. It may be that the concentration ratio of the chemical molecule in the first solution:co-former in the third solution is from 10:1 to 1:10. It may be that the concentration ratio of the co-former in the second solution:co-former in the third solution is from 15:1 to 1:15. It may be that the concentration ratio of the co-former in the second solution:co-former in the third solution is from 12:1 to 1:12. It may be that the concentration ratio of the co-former in the second solution:co-former in the third solution is from 10:1 to 1:10.

It may be that the concentration (mg/mL) ratio of the chemical molecule in the first solution:co-former in the fourth solution is from 15:1 to 1:15. It may be that the concentration ratio of the chemical molecule in the first solution:co-former in the fourth solution is from 12:1 to 1:12. It may be that the concentration ratio of the chemical molecule in the first solution:co-former in the fourth solution is from 10:1 to 1:10. It may be that the concentration ratio of the co-former in the second solution:co-former in the fourth solution is from 15:1 to 1:15. It may be that the concentration ratio of the co-former in the second solution:co-former in the fourth solution is from 12:1 to 1:12. It may be that the concentration ratio of the co-former in the second first solution:co-former in the fourth solution is from 10:1 to 1:10. It may be that the concentration ratio of the co-former in the third solution:co-former in the fourth solution is from 15:1 to 1:15. It may be that the concentration ratio of the co-former in the third solution:co-former in the fourth solution is from 12:1 to 1:12. It may be that the concentration ratio of the co-former in the third first solution:co-former in the fourth solution is from 10:1 to 1:10.

The step of allowing the crystal to form typically comprises leaving the droplet for a period of time, e.g. until crystal formation is observed. Thus, the method may comprise the step of looking for signs of crystal formation, e.g. using the naked eye or, more typically, using optical microscopy. The droplet may be left for a period of time from 2 hours to 31 days, e.g. a period of time from 1 day to 14 days.

The droplet may be left at room temperature and atmospheric pressure. Alternatively, the droplet may be left at a temperature below room temperature, e.g. a temperature in the range from −25° C. to 15° C. Alternatively, the droplet may be left at a temperature above room temperature, e.g. a temperature in the range from 30° C. to 60° C.

The co-crystal may be the only solid formed. More typically, the co-crystal that is formed is one of a plurality of co-crystals that are formed or the co-crystal is formed alongside other solid forms, e.g. amorphous solids, polycrystalline materials, single component crystals of either the molecule or the co-former.

The method may comprise the step of recovering the co-crystal. Where, the droplet was in a sealed vessel, this step may comprise unsealing the vessel and removing the crystal from the vessel.

The method may comprise the step of performing single crystal X-ray crystallography on the co-crystal.

The method of the first aspect may be a method of determining the structure of a chemical (e.g. organic) molecule, or salt thereof, the method comprising: forming a plurality of droplets according to the first aspect; allowing crystals to form from said plurality of droplets; optionally looking for signs of crystal formation; recovering any co-crystals that are formed; performing single crystal X-ray crystallography on the co-crystals that are formed; and using the results of the single crystal X-ray crystallography to determine the structure of the chemical (e.g. organic) molecule, or salt thereof; wherein at least two of the plurality of droplets are different.

It may be that at least three of the plurality of droplets are different. It may be that at least five of the plurality of droplets are different. It may be that no one of the plurality of droplets is the same as any other one of the plurality of droplets. Thus, the plurality of droplets may each be different.

It may be that each droplet is formed in a respective well of a multi-well plate. The droplets may be formed by a robot.

In this method, it is not necessarily the case that co-crystals form from every droplet. In some droplets, no crystal at all may form. In some droplets, it may be that only single component crystals of either the molecule or the co-former form. It may be that a co-crystal forms in only a subset of the droplets. It may be that a co-crystal forms in only one of the droplets. Even a single co-crystal may be sufficient to determine the structure of the organic molecule.

The plurality of droplets will typically comprise the same chemical (e.g. organic) molecule, or salt thereof. They may differ in any other aspect of the droplet's composition. Examples include: the oil of the second liquid; the solvent or solvents of which the first liquid is comprised; where more than one solvent is present in the first liquid, the relative amounts of those solvents in the first liquid; the concentration of the chemical (e.g. organic) molecule or salt thereof in the first liquid; identity of the co-former(s); the concentration of the co-former(s) or salt(s) thereof in the first liquid; the relative proportions of the chemical (e.g. organic) molecule or salt thereof and the co-former(s) or salt(s) thereof; the amount of the co-former(s) or salt(s) thereof; the amount of the chemical (e.g. organic) molecule, or salt thereof; the amount of the first liquid; the ratio of the second liquid to the first liquid; the presence or absence of an antisolvent; the identity of the antisolvent, if present; the presence or absence of a seeding material; the identity of the seeding material, if present; etc.

It may be that the at least two of the plurality of droplets differ in at least one condition selected from: the oil of the second liquid; the solvent or solvents of which the first liquid is comprised; the concentration of the chemical (e.g. organic) molecule or salt thereof in the first liquid; the concentration of the co-former(s) or salt(s) thereof in the first liquid; the relative proportions of the chemical (e.g. organic) molecule or salt thereof and the co-former(s) or salt(s) thereof; the amount of the co-former(s) or salt(s) thereof; the amount of the chemical (e.g. organic) molecule, or salt thereof.

The method of determining the structure of a chemical (e.g. organic) molecule, or salt thereof, may additionally comprise forming one or more droplets not according to the first aspect. The formation of the one or more droplets not according to the first aspect may occur before the formation of a plurality of droplets according to the first aspect. The formation of one or more droplets not according to the first aspect may occur at the same time as the formation of a plurality of droplets according to the first aspect. The formation of one or more droplets not according to the first aspect may occur after the formation of a plurality of droplets according to the first aspect. The formation of one or more droplets not according to the first aspect typically occurs at some time before the step of allowing the crystals to form.

It may be that at least one of the one or more droplets not according to the first aspect do not comprise an oil. It may be that at least one of the one or more droplets not according to the first aspect that do not comprise an oil comprise a high boiling point solvent, e.g. a solvent with a boiling point greater than 125° C.

The method of determining the structure of a chemical (e.g. organic) molecule, or salt thereof, that additionally comprises forming one or more droplets not according to the first aspect may also comprise subjecting the one or more droplets not according to the first aspect to the subsequent method steps (i.e., every step after the first step) referred to above.

The method of the first aspect may be a method of screening for co-crystals of a chemical (e.g. organic) molecule, or salt thereof, the method comprising: forming a plurality of droplets according to the first aspect; allowing crystals to form from said plurality of droplets; optionally looking for signs of crystal formation; recovering any co-crystals that are formed; performing single crystal X-ray crystallography on the co-crystals that are formed; and optionally, comparing the results of the single crystal X-ray crystallography for each co-crystal to the results of the single crystal X-ray crystallography for each other crystal and/or to known co-crystals of the chemical (e.g. organic) molecule, or salt thereof; wherein at least two of the plurality of droplets are different.

The method of screening for co-crystals of a chemical (e.g. organic) molecule, or salt thereof, may be a method of screening for crystallisation conditions suitable for forming a co-crystal of the chemical molecule, or salt thereof.

It may be that at least three of the plurality of droplets are different. It may be that at least five of the plurality of droplets are different. It may be that no one of the plurality of droplets is the same as any other one of the plurality of droplets. Thus, the plurality of droplets may each be different.

It may be that each droplet is formed in a respective well of a multi-well plate. The droplets may be formed by a robot.

In this method, it is not necessarily the case that co-crystals form from every droplet. In some droplets, no crystal at all may form. In some droplets, it may be that only single component crystals of either the molecule or the co-former form. It may be that a co-crystal forms in only a subset of the droplets. It may be that a co-crystal forms in only one of the droplets. Even a single co-crystal may be sufficient to determine the structure of the organic molecule.

The plurality of droplets will typically comprise the same chemical (e.g. organic) molecule, or salt thereof. They may differ in any other aspect of the droplet's composition. Examples include: the oil of the second liquid; the solvent or solvents of which the first liquid is comprised; where more than one solvent is present in the first liquid, the relative amounts of those solvents in the first liquid; the concentration of the chemical (e.g. organic) molecule or salt thereof in the first liquid; identity of the co-former(s); the concentration of the co-former(s) or salt(s) thereof in the first liquid; the relative proportions of the chemical (e.g. organic) molecule or salt thereof and the co-former(s) or salt(s) thereof; the amount of the co-former(s) or salt(s) thereof; the amount of the chemical (e.g. organic) molecule, or salt thereof; the amount of the first liquid; the ratio of the second liquid to the first liquid; the presence or absence of an antisolvent; the identity of the antisolvent, if present; the presence or absence of a seeding material; the identity of the seeding material, if present; etc.

It may be that the at least two of the plurality of droplets differ in at least one condition selected from: the oil of the second liquid; the solvent or solvents of which the first liquid is comprised; the concentration of the chemical (e.g. organic) molecule or salt thereof in the first liquid; the concentration of the co-former(s) or salt(s) thereof in the first liquid; the relative proportions of the chemical (e.g. organic) molecule or salt thereof and the co-former(s) or salt(s) thereof; the amount of the co-former(s) or salt(s) thereof; the amount of the chemical (e.g. organic) molecule, or salt thereof.

### BRIEF DESCRIPTION OF THE DRAWINGS

Embodiments of the invention are further described hereinafter with reference to the accompanying drawings, in which:

FIG. 1 shows the crystal structure of caffeine-6-hydroxy-2-napthoic acid co-crystal.

FIG. 2 shows the crystal structure of 4,4-bipyridine-5-chlorosalicylic acid co-crystal.

FIG. 3 shows the crystal structure of caffeine-5-chlorosalicylic acid co-crystal.

FIG. 4 shows the crystal structure of carbamazepine-quinol co-crystal.

FIG. 5 shows the crystal structure of 3,5-dinitrobenzoic acid-3-methylbenzoic acid-iso-nicotinamide co-crystal.

FIG. 6 shows the crystal structures of co-crystals 1 to 37 crystallised in Example 6.

### DETAILED DESCRIPTION

The term ‘organic medium’ is intended to mean a liquid that comprises an organic component, i.e. an organic component other than the chemical molecule that is being crystallised. The organic component may be an organic solvent, i.e. an uncharged organic molecule, or it may comprise organic components which comprise organic ions, e.g. ionic liquids or deep eutectic solvents. The components of the organic medium will typically be miscible at the relative concentrations at which they are used, but the organic medium may also take other forms, e.g. an emulsion.

The term ‘organic solvent’ typically refers to organic molecules having no net charge that form a liquid at 25° C. and 1 atm. Typically, they have a molecular mass in the range 30 gmol⁻¹ to 150 gmol⁻¹, e.g. 40 gmol⁻¹ to 100 gmol⁻¹.

The term ‘oil’ typically refers to an inert chemical substance that is a liquid at 25° C. and 1 atm. Oils are typically immiscible with water. Typically, an oil is non-volatile. Typically, an oil has a boiling point above 250° C. Typically, an oil has a molecular weight above 500 gmol^{31 1}.

‘Organic molecules’ typically include at least one carbon-carbon covalent bond and typically comprise only atoms selected from H, C, N, O, P, S, F, Cl, Br and I, said atoms being bonded together by covalent bonds.

Organometallic molecules typically comprise organic portions (i.e. portions include at least one carbon-carbon covalent bond and comprising only atoms selected from H, C, N, O, P, S, F, Cl, Br and I) covalently or datively bonded to metal atoms.

A co-former is any additional component that forms part of the same crystal lattice as the primary component. In the context of the present invention, the primary component is the chemical molecule that is dissolved in the first solution. The second solution, and optionally, the third, fourth, and any further solution(s), comprise a co-former that will form part of the same crystal lattice as the chemical molecule. Suitable co-formers for use in the present invention will typically be an organic (e.g. small organic) molecules. The co-former(s) may be in the form of a salt. The co-former(s) may be charged.

It may be that the co-crystal comprises a single molecule of the chemical molecule, or salt thereof, per crystal structure unit cell. It may be that co-crystal comprises less than one molecule of the chemical molecule, or salt thereof, (e.g. 0.5 molecules) per crystal structure unit cell. It may be that co-crystal comprises more than one molecule of the chemical molecule, or salt thereof, (e.g. 1.5, 2, 2.5 or 3 molecules) per crystal structure unit cell.

It may be that the co-crystal comprises a single molecule of the co-former, or salt thereof, per crystal structure unit cell. It may be that co-crystal comprises less than one molecule of the co-former, or salt thereof, (e.g. 0.5 molecules) per crystal structure unit cell. It may be that co-crystal comprises more than one molecule of the co-former, or salt thereof, (e.g. 1.5, 2, 2.5 or 3 molecules) per crystal structure unit cell.

Typically, the co-former(s) will be regarded as biologically inert (or inactive) such as molecules from the US Food and Drug Administration's GRAS (generally regarded as safe) list. A list of such molecules can be found in the GRAS Substances Database (https://www.cfsana psexternal.fda.gov/scripts/fdcc/?set=SCOGS). Exemplary co-formers include: carboxylic acids (e.g. oxalic acid, malonic acid, maleic acid, glutaric acid, benzoic acid), phenolics (e.g. phenol, t-butyl-phenol), amino acids (e.g. glycine, valine, serine), sugars (e.g. glucose, maltose). Alternatively however, the co-former(s) may be biologically active, such as salicylic acid and caffeine. The co-former(s) may be a drug. When two or more co-formers are present, it may be that one co-former is a drug and the other(s) are regarded as biologically inert.

The term “drug” is intended to cover any molecule that may be administered to a biological organism, e.g. a human being, to restore, correct or modify a physiological function by exerting a pharmacological action in order to treat or prevent a disease or illness. The term “drug” is also intended to cover diagnostic agents. A “prodrug” is a compound that does not exert the intended pharmacological action itself but is metabolised in the body to produce an active therapeutic drug. Typically, the drug or prodrug will be an organic compound, or a compounds comprising an organic moiety, e.g. an organometallic compound. The drug or prodrug will have received licensing from a regulatory body, such as the MHRA, EMA or FDA, to treat a certain illness or disease. A comprehensive list of drugs and prodrugs can be found in The Merck Index Online (https://www.rsc.org/merck-index).

The prefix ‘perfluoro-’ and the term ‘perfluorinated’ relate to organic molecules, including those containing heteroatoms, that contain only carbon-fluorine bonds, carbon-carbon bonds and carbon-heteroatom bonds, i.e. do not contain any C—H bonds.

The term “syringe” is intended to cover any apparatus (e.g. vessel, machine, etc), that can both take in (to the apparatus) and eject a liquid solution. The syringe may be an air displacement type syringe. The syringe may be a pipette. The syringe may be operated by a robot.

For the purpose of this specification, a droplet is a body of liquid that is in contact with a single surface of a support, wherein the surface is such that the body of liquid is not subject to capillarity. The cohesive forces of the liquid(s) in the body of liquid outweigh the adhesive forces of said liquid(s) to the surface that the body of liquid is in contact with (thereby forming a droplet). The term is therefore intended to exclude (flat) sheets of liquid in which the adhesive forces of the liquid(s) to the support surface outweigh the cohesive forces of the liquid(s). The droplets of the invention may be in contact with a flat, convex or concave support surface. That support surface may form the base of a vessel or the top of a vessel. The term is intended to exclude bodies of liquid that are simultaneously in contact with the sides and base of a vessel and bodies of liquid that are in contact with a single continuous surface of a tube in which the liquid would be subject to capillarity (e.g. a capillary tube).

Without wishing to be bound by theory, the benefit of using droplets is that it allows the use of very low volumes of organic medium and oil and are therefore suitable for forming crystals from very low amounts of analyte. Without wishing to be bound by theory, the low volume also minimises the convection currents within the sample and reduces the number of nucleation sites and it is believed that this may also benefit the formation of crystals on a small scale.

The term ‘encapsulated’ is intended to mean ‘completely encapsulated’. The first liquid is encapsulated by the second liquid where all of the surface of the first liquid that is not in contact with the support surface are coated with the second liquid.

A saturated solution (of a solute) has the same solute concentration as a solution that is in equilibrium with undissolved solute (at room temperature and atmospheric pressure). A near saturated solution has a solute concentration that is no more than 20% below that of a saturated solution of the same solute in the same solvent. A near saturated solution may have a solute concentration that is no more than 10% below that of a saturated solution of the same solute in the same solvent or no more than 5% below that of a saturated solution of the same solute in the same solvent. In a practical context, a near saturated solution is typically obtained by adding the solvent dropwise or portion wise to a solid sample of the solute until the solid is completely dissolved. At the point that the solute is completely dissolved, the concentration of the solute in the solvent will be close to but slightly less than the concentration of a saturated solution of the same solute in the same solvent.

The chemical molecule may be present in the form of a salt, e.g. a pharmaceutically acceptable salt. For use in medicine, the salts of the compounds of this invention refer to “pharmaceutically acceptable salts.” FDA approved pharmaceutical acceptable salt forms (Ref. International J. Pharm. 1986, 33, 201-217; J. Pharm. Sci., 1977, January, 66 (1)) include pharmaceutically acceptable acidic/anionic or basic/cationic salts. Suitable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, maleate, mandelate, mesylate, methylbromide, methylnitrate, methylsulfate, mucate, napsylate, nitrate, pamoate, pantothenate, phosphate, diphospate, polygalacturonate, salicylate, stearate, subacetate, succinate, sulfate, tannate, tartrate, teoclate, tosylate and triethiodide. Suitable basic/cationic salts include, and are not limited to aluminium, benzathine, calcium, chloroprocaine, choline, diethanolamine, ethylenediamine, lithium, magnesium, potassium, procaine, sodium and zinc. In certain embodiments, however, the chemical molecule may be present not in the form of a salt.

Cambridge Structural Database (CSD) refcodes can be searched via the Cambridge Crystallographic Data Centre's website (https://www.ccdc.cam.ac.uk/structures/).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

An illustrative method of forming a crystal of a chemical molecule according to the present invention is described below.

### General Procedure

### Stock Solution Preparation Methods One, Two and Three

Stock solutions of the compound and the appropriate co-former were freshly prepared for each crystallisation experiment. Samples were weighed (~2-4 mg) into screw top vials and dissolved in one of 12 solvents (dimethyl sulfoxide (DMSO), dimethyl formamide (DMF), methanol (MeOH), 2,2,2-trifluoroethanol (2,2,2-TFE), toluene, 1,2-dichloroethane (DCE), 2-methyltetrahydrofuran (2-MeTHF), 1,4-dioxane, ethyl acetate (EtOAc), acetonitrile (MeCN), 4-methyl-2-pentanone (4-MP), and nitromethane (MeNO₂)), through added portion wise solvent addition until a near saturated solution was formed.

### Method One

Four oils (200 nL each) were then dispensed using a SPT Labtech Mosquito® liquid-handling robot onto a SWISSSCI LCP glass plate with a 100 μm spacer (aspirate 1.0 mm min⁻¹, dispense 1.0 mm min⁻¹).

After which total of 150 nL of compound and co-former stock solution was injected into each oil droplet (aspirate 20 mm min⁻¹, dispense 20 mm min⁻¹) with varied ratios across the plate: 50 nL:100 nL, 75 nL: 75 nL, and 100 nL: 50 nL. This was achieved through pick-up of the appropriate volume of compound stock solution, followed by the sequential pick-up of the appropriate volume of one or more co-former stock solutions into the same needle (so that the needle contains each of the picked-up solutions), followed by injection of the solutions into each oil droplet. Typically, the solutions are injected into the droplet within 5 to 30 seconds after being picked-up.

Plates were then sealed with a glass cover slip and left for 7 days before inspection for crystallisation.

### Method Two

Four oils (200 nL each) were then dispensed using a SPT Labtech Mosquito® liquid-handling robot onto a SWISSSCI LCP glass plate with a 100 μm spacer (aspirate 1.0 mm min⁻¹, dispense 1.0 mm min⁻¹).

50, 75 or 100 nL of compound stock solution was picked up and injected into a well (within a 96-well glass coated V-shaped well parent plate) containing the co-former stock solution (10 μL). The resulting sample was mixed by the robot by pick up and injection of 1000 nL of solution over 5 cycles. After which 150 nL of the mixed compound/co-former solution was picked up and injected into each oil droplet (aspirate 20 mm min⁻¹, dispense 20 mm min⁻¹).

Plates were then sealed with a glass cover slip and left for 7 days before inspection for crystallisation.

### Method Three

A total of 150 nL of compound and co-former stock solution was injected onto a SWISSSCI LCP glass plate with a 100 μm spacer (aspirate 20 mm min⁻¹, dispense 20 mm min⁻¹) with varied ratios across the plate: 50 nL:100 nL, 75 nL: 75 nL, and 100 nL: 50 nL. This was achieved through pick-up of the appropriate volume of compound stock solution, followed by the pick-up of the appropriate volume of co-former stock solution into the same needle, and injection of the combined sample onto the plate, followed by mixing of both components by the robot via pick up and injection (150 nL, 1 cycle).

Four oils (200 nL each) were then dispensed (aspirate 1.0 mm min⁻¹, dispense 1.0 mm min⁻¹), encapsulating the sample.

Plates were then sealed with a glass cover slip and left for 7 days before inspection for crystallisation.

### Method Four

Stock solutions containing both the compound and the co-former were freshly prepared for each crystallisation experiment. Samples were weighed (the compound and the co-former in different ratios, to a total of −8 mg) into screw top vials and dissolved in one of 12 solvents (dimethyl sulfoxide (DMSO), dimethyl formamide (DMF), methanol (MeOH), 2,2,2-trifluoroethanol (2,2,2-TFE), toluene, 1,2-dichloroethane (DCE), 2-methyltetrahydrofuran (2-MeTHF), 1,4-dioxane, ethyl acetate (EtOAc), acetonitrile (MeCN), 4-methyl-2-pentanone (4-MP), and nitromethane (MeNO₂)), through added portion wise solvent addition until a near saturated solution was formed, followed by sonication.

Four oils (200 nL each) were then dispensed using a SPT Labtech Mosquito® liquid-handling robot onto a SWISSSCI LCP glass plate with a 100 μm spacer (aspirate 1.0 mm min⁻¹, dispense 1.0 mm min⁻¹).

After which 150 nL of the mixed compound/co-former solutions were injected into each oil droplet (20 mm min⁻¹, dispense 20 mm min⁻¹). Plates were then sealed with a glass cover slip and left for 7 days before inspection for crystallisation.

### Example 1: Caffeine/6-hydroxy-2-napthoic Acid Co-Crystal

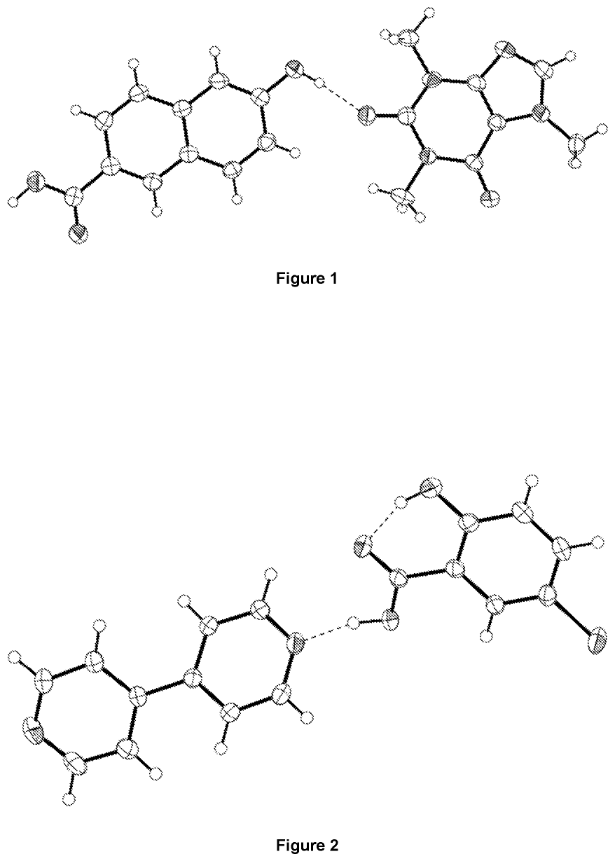

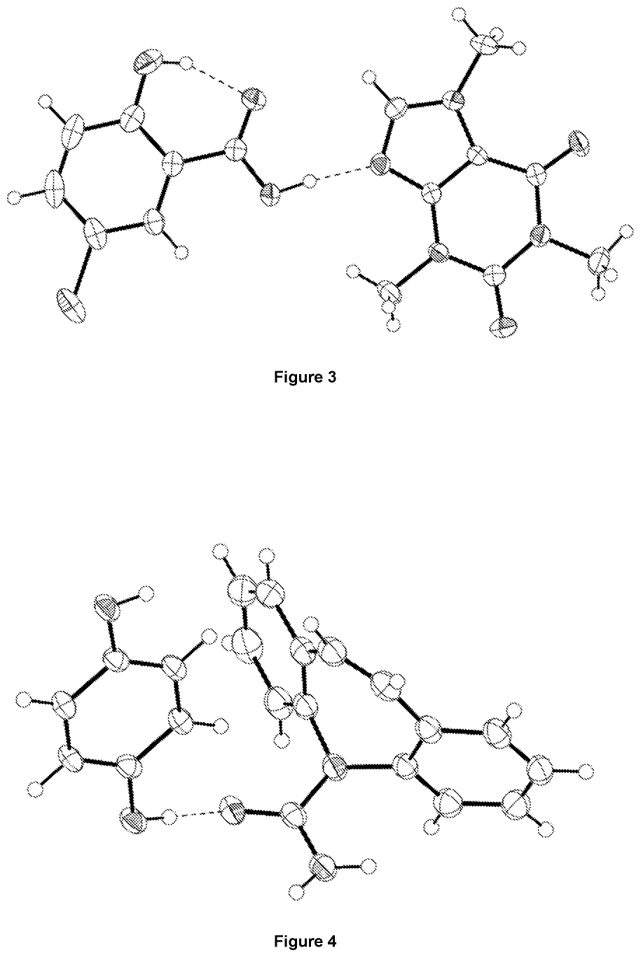

Using method one, caffeine and 6-hydroxy-2-napthoic acid were co-crystallised, and a crystal of suitable size and quality for x-ray powder diffraction (XRPD) analysis was obtained within 1 week after plate setup. A co-crystal with one molecule of caffeine and 6-hydroxy-2-napthoic acid in the asymmetric unit was obtained. See FIG. 1.

### Example 2: 4,4-Bipyridine/5-chlorosalicylic Acid Co-Crystal

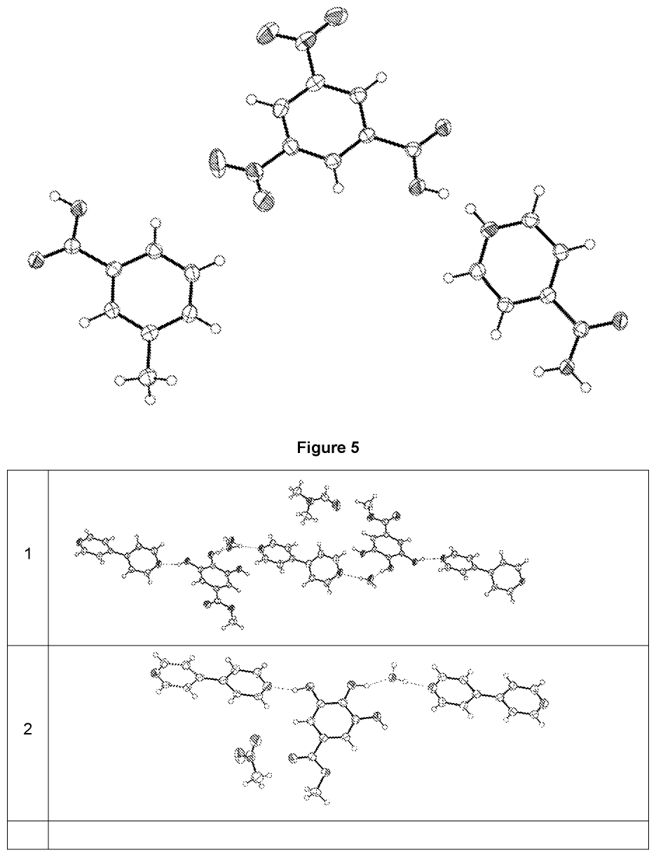

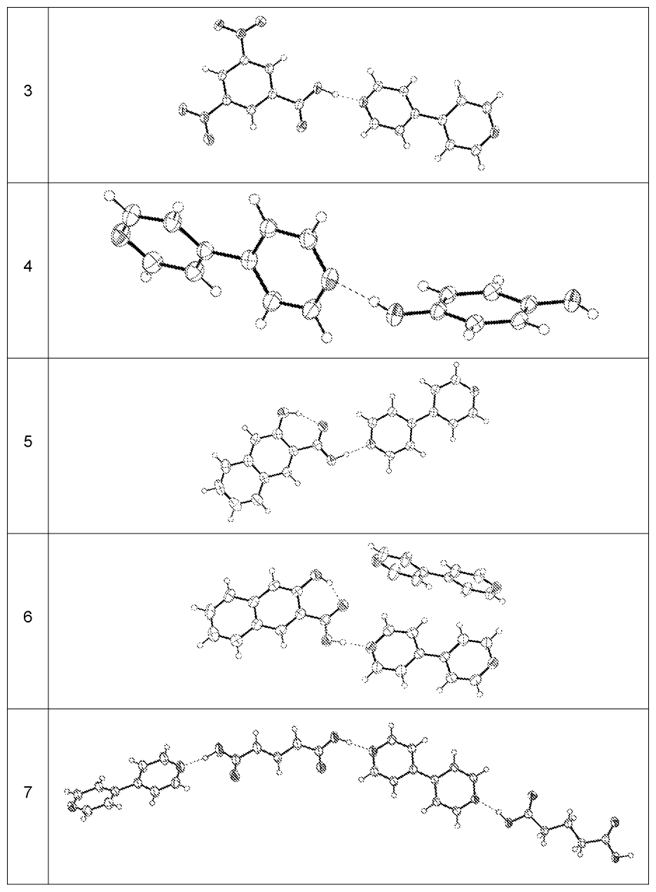

Using method two, 4,4-bipyridine and 5-chlorosalicylic acid were co-crystallised, and a crystal of suitable size and quality for XRPD analysis was obtained within 1 week after plate setup. A co-crystal with one molecule of 4,4-bipyridine and one molecule of 5-chlorosalicylic acid in the asymmetric unit was obtained. See FIG. 2.

### Example 3: Caffeine/5-chlorosalicylic Acid Co-Crystal

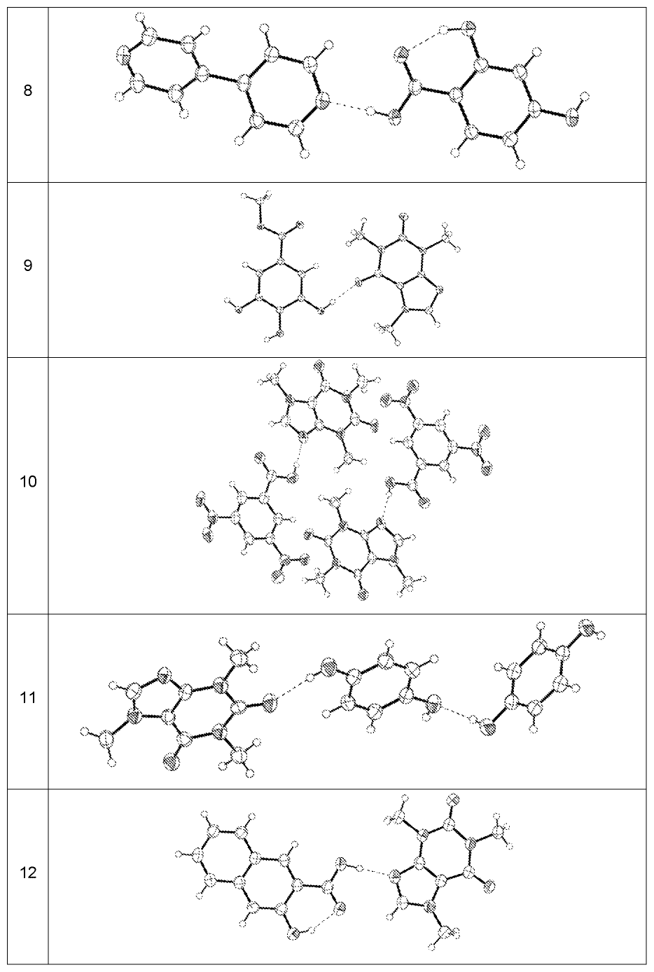

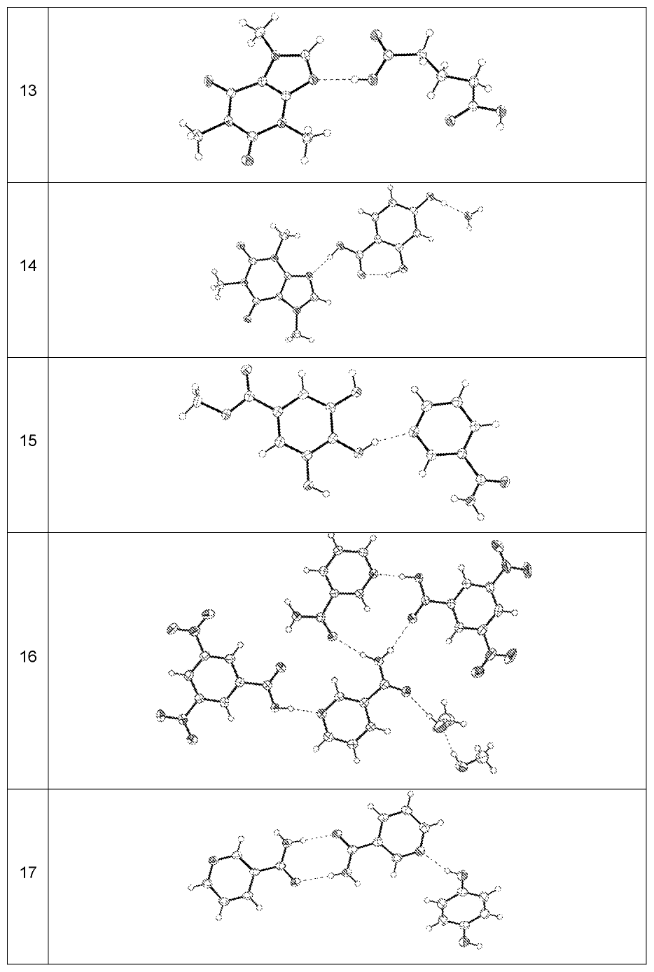

Using method three, caffeine and 5-chlorosalicylic acid were co-crystallised, and a crystal of suitable size and quality for XRPD analysis was obtained within 1 week after plate setup. A co-crystal with one molecule of caffeine and one molecule of 5-chlorosalicylic acid in the asymmetric unit was obtained. See FIG. 3.

### Example 4: Carbamazepine/Quinol Acid Co-Crystal

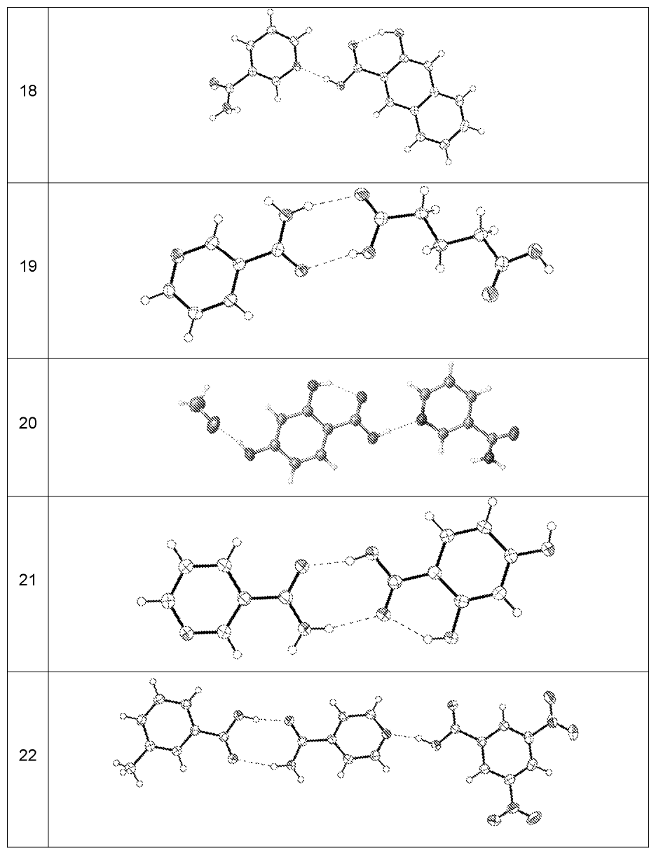

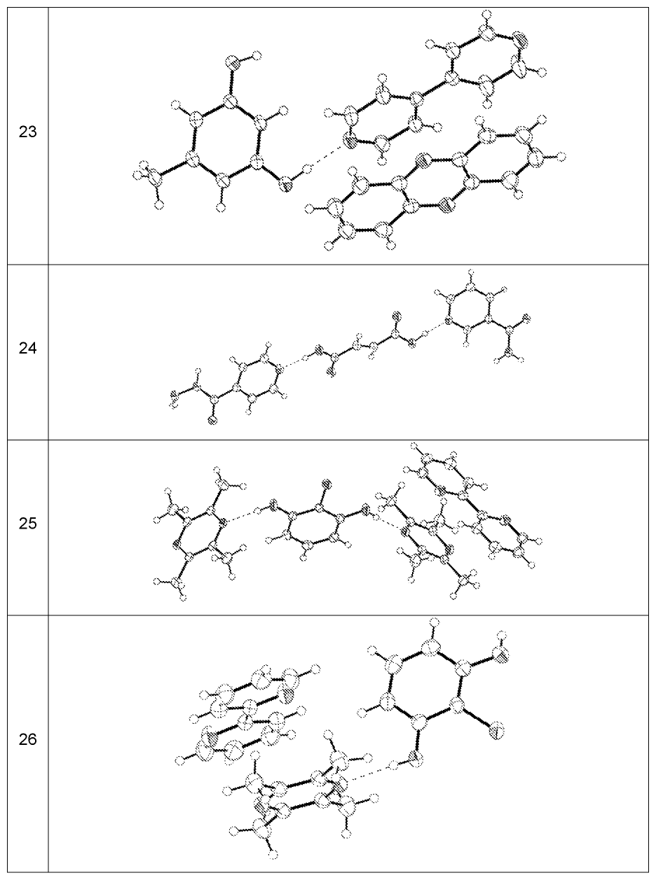

Using method four, carbamazepine and quinol acid were co-crystallised, and a crystal of suitable size and quality for XRPD analysis was obtained within 1 week after plate setup. A co-crystal with one molecule of carbamazepine and one molecule of quinol in the asymmetric unit was obtained. See FIG. 4.

### Example 5: 3,5-Dinitrobenzoic Acid/3-Methylbenzoic Acid/Iso-Nicotinamide Co-Crystal

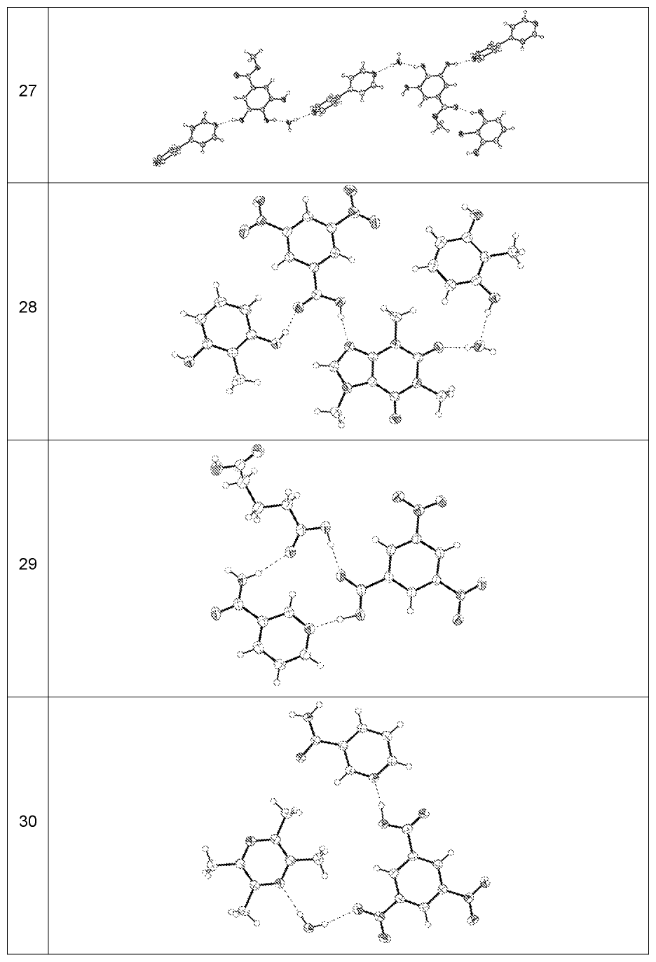

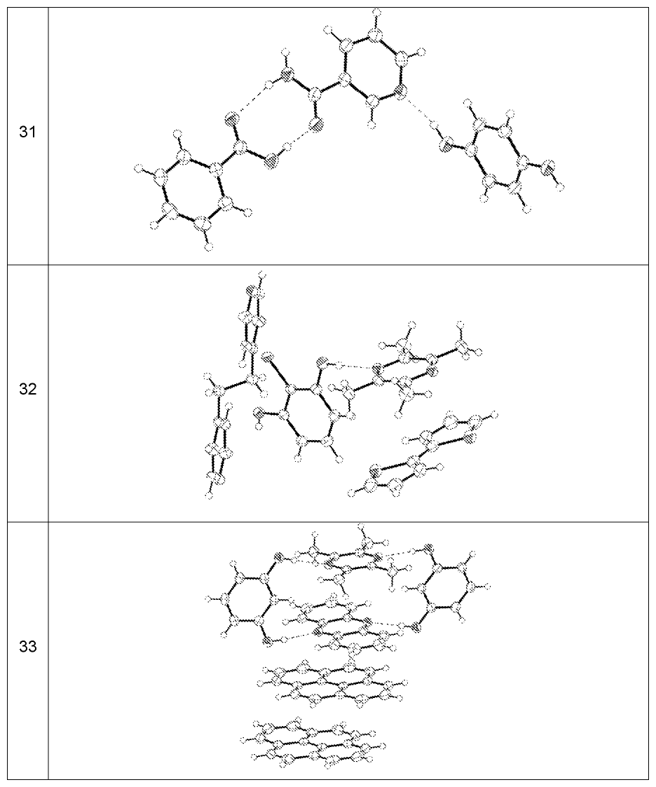

Using method one, 3,5-dinitrobenzoic acid, 3-methylbenzoic acid and iso-nicotinamide were co-crystallised, and a crystal of suitable size and quality for XRPD analysis was obtained within 1 week after plate setup. A co-crystal with one molecule of 3,5-dinitrobenzoic acid, one molecule of 3-methylbenzoic acid and one molecule of iso-nicotinamide in the asymmetric unit was obtained. See FIG. 5.

### Example 6: Further Binary, Ternary and Quaternary Co-Crystals

Using method one, a number of compounds and co-formers were co-crystallised as binary, ternary or quaternary co-crystals.

Stock solutions of the compound and the appropriate co-former were freshly prepared for each crystallisation experiment. Samples were weighed (~2 mg) into screw top vials and dissolved in one of 4 solvents (dimethyl formamide (DMF), methanol (MeOH), 1,4-dioxane, and nitromethane (MeNO₂)), through portion wise solvent addition until a near saturated solution was formed. Samples were then dissolved in either 12, 24, 48 or 89 mL of the solvent to provide 167, 83, 42 or 21 mg mL⁻¹ solutions, respectively (see Table 1).

TABLE 1

| | | Conc. | | | Conc. | | | Conc. |
|---|---|---|---|---|---|---|---|---|
| | | (mg | | | (mg | | | (mg |
| | Solvent | mL⁻¹) | | Solvent | mL⁻¹) | | Solvent | mL⁻¹) |
| 1,2-bis(4- | DMF | 167 | 4,4′ - | DMF | 167 | nicotinic | DMF | 83 |
| pyridyl) | MeOH | 167 | bipyridine | MeOH | 167 | acid | MeOH | 83 |
| ethane | 1,4- | 167 | | 1,4- | 83 | | 1,4- | 83 |
| | Dioxane | | | Dioxane | | | Dioxane | |
| | MeNO₂ | 167 | | MeNO₂ | 167 | | MeNO₂ | 42 |
| 2- | DMF | 167 | benzoic | DMF | 167 | Orcinol | DMF | 167 |
| bromoresorcinol | MeOH | 83 | acid | MeOH | 167 | | MeOH | 167 |
| | 1,4- | 167 | | 1,4- | 167 | | 1,4- | 83 |
| | Dioxane | | | Dioxane | | | Dioxane | |
| | MeNO₂ | 167 | | MeNO₂ | 42 | | MeNO₂ | 83 |
| 2- | DMF | 167 | Caffeine | DMF | 21 | oxalic | DMF | 83 |
| chlororesorciol | MeOH | 83 | | MeOH | 21 | acid | MeOH | 83 |
| | 1,4- | 167 | | 1,4- | 42 | | 1,4- | 83 |
| | Dioxane | | | Dioxane | | | Dioxane | |
| | MeNO₂ | 83 | | MeNO₂ | 83 | | MeNO₂ | 21 |
| 2- | DMF | 167 | fumaric | DMF | 167 | Phenazine | DMF | 83 |
| methylresorcinol | MeOH | 167 | acid | MeOH | 21 | | MeOH | 42 |
| | 1,4- | 83 | | 1,4- | 42 | | 1,4- | 83 |
| | Dioxane | | | Dioxane | | | Dioxane | |
| | MeNO₂ | 83 | | MeNO₂ | 42 | | MeNO₂ | 83 |
| 2,2′ - | DMF | 83 | glutaric | DMF | 83 | Pyrene | DMF | 83 |
| bipyridine | MeOH | 167 | acid | MeOH | 167 | | MeOH | 42 |
| | 1,4- | 167 | | 1,4- | 83 | | 1,4- | 83 |
| | Dioxane | | | Dioxane | | | Dioxane | |
| | MeNO₂ | 167 | | MeNO₂ | 83 | | MeNO₂ | 83 |
| 2,2′ - | DMF | 167 | Isoniazid | DMF | 167 | Quinol | DMF | 83 |
| bithiophene | MeOH | 167 | | MeOH | 167 | | MeOH | 167 |
| | 1,4- | 167 | | 1,4- | 167 | | 1,4- | 42 |
| | Dioxane | | | Dioxane | | | Dioxane | |
| | MeNO₂ | 42 | | MeNO₂ | 42 | | MeNO₂ | 42 |
| 2,4- | DMF | 167 | Isonicotinamide | DMF | 167 | Resorcinol | DMF | 167 |
| dihydroxybenzoic | MeOH | 167 | | MeOH | 83 | | MeOH | 167 |
| acid | 1,4- | 83 | | 1,4- | 83 | | 1,4- | 83 |
| | Dioxane | | | Dioxane | | | Dioxane | |
| | MeNO₂ | 83 | | MeNO₂ | 21 | | MeNO₂ | 83 |
| 3-hydroxy-2- | DMF | 83 | methyl gallate | DMF | 167 | Tetramethyl- | DMF | 167 |
| napthoic acid | MeOH | 167 | | MeOH | 167 | pyrazine | MeOH | 167 |
| | 1,4 | 83 | | 1,4- | 83 | | 1,4- | 83 |
| | Dioxane | | | Dioxane | | | Dioxane | |
| | MeNO₂ | 21 | | MeNO₂ | 21 | | MeNO₂ | 167 |
| 3,5- | DMF | 167 | Nicotinamide | DMF | 83 | toluic | DMF | 167 |
| dinitrobenzoic | MeOH | 167 | | MeOH | 83 | acid | MeOH | 167 |
| acid | 1,4- | 83 | | 1,4- | 42 | | 1,4- | 83 |
| | Dioxane | | | Dioxane | | | Dioxane | |
| | MeNO₂ | 83 | | MeNO₂ | 42 | | MeNO₂ | 83 |
| 4- | DMF | 167 | | | | | | |
| hydroxybenz | MeOH | 167 | | | | | | |
| amide | 1,4- | 21 | | | | | | |
| | Dioxane | | | | | | | |
| | MeNO₂ | 21 | | | | | | |

### Binary Co-Crystallisation

Binary co-crystallisation experiments were set up utilising 2 plates (BP1 and BP2) covering 2 solvents per plate and ratios 2:1, 1:1 and 1:2.

### Ternary Co-Crystallisation

Ternary co-crystallisation experiments were set up utilising 3 plates (TP1, TP2 and TP3) covering 4 solvents per plate and ratios 2:1:1, 1:2:1, and 1:1:2 (TP1), 2:2:1, 2:1:2 and 1:2:2 (TP2) and 1:1:1 (TP3).

### Quaternary Co-Crystallisation

Quaternary co-crystallisation experiments were set up utilising 1 plate (QP1) covering 4 solvents per plate and ratio 1:1:1:1.

### Compound and Co-Former Combinations

The attempted compound and co-former combination co-crystallisations are provided in Table 5.

TABLE 5

| Binary Combinations | Ternary Combinations |
|---|---|
| 4,4′-bipyridine: 2,4-dihydroxybenzoic acid | 4,4′-bipyridine: 3-hydroxy-2-naphthoic acid: |
| | 1,2-bis(4-pyridyl)ethane |
| 4,4′-bipyridine: 3,5-dinitrobenzoic acid | 4,4′-bipyridine: 3-hydroxy-2-naphthoic acid: |
| | 2,2′-bipyridine |
| 4,4′-bipyridine: 3-hydroxy-2-napthoic acid | 4,4′-bipyridine: 3-hydroxy-2-naphthoic acid: |
| | phenazine |
| 4,4′-bipyridine: glutaric acid | 4,4′-bipyridine: 3-hydroxy-2-naphthoic acid: |
| | tetramethylpyrazine |
| 4,4′-bipyridine: methyl gallate | 4,4′-bipyridine: glutaric acid: 3,3′- |
| | thiodipropionic acid |
| 4,4′-bipyridine: quinol | 4,4′-bipyridine: glutaric acid: 5- |
| | nitroisophthalic acid |
| caffeine: 2,4-dihydroxybenzoic acid | 4,4′-bipyridine: glutaric acid: phenazine |
| caffeine: 3,5-dinitrobenzoic acid | 4,4′-bipyridine: glutaric acid: |
| | tetramethylpyrazine |
| caffeine: 3-hydroxy-2-naphthoic acid | 4,4′-bipyridine: glutaric acid: trimesic acid |
| caffeine: glutaric acid | 4,4′-bipyridine: orcinol: phenazine |
| caffeine: methyl gallate | caffeine: quinol: 1,4-dithiane-2,5-diol |
| caffeine: quinol | caffeine: quinol: 2,3,5-tetramethylbenzene- |
| | 1,4-diol |
| nicotinamide: 2,4-dihydroxybenzoic acid | caffeine: quinol: 4-hydroxybenzoic acid |
| nicotinamide: 3,5-dinitrobenzoic acid | caffeine: quinol: resorcinol |
| nicotinamide: 3-hydroxy-2-napthoic acid | caffeine: quinol: tetramethylpyrazine |
| nicotinamide: glutaric acid | nicotinamide: 3,5-dinitrobenzoic acid: |
| | glutaric acid |
| nicotinamide: methyl gallate | nicotinamide: 3,5-dinitrobenzoic acid: |
| | tetramethylpyrazine |
| nicotinamide: quinol | nicotinamide: fumaric acid: isoniazid |
| | nicotinamide: quinol: benzoic acid |
| | nicotinamide: quinol: isonicotinamide |
| | nicotinamide: quinol: nicotinic acid |
| | tetramethylpyrazine: 2,2′-bipyridine: 2- |
| | chlororesorcinol |
| | toluic acid: isonicotinamide: 3,5- |
| | dinitrobenzoic acid |

Quaternary Combinations
2-bromoresorcinol: tetramethylpyrazine: 2,2′-bipyridine: 1,2-bis(4-pyridyl)ethane
2-chlorolresorcinol: tetramethylpyrazine: 2,2′-bithiophene: 1,2-bis(4-pyridyl)ethane
2-chlororesorcinol: tetramethylpyrazine: 2,2′-bipyridine: 1,2-bis(4-pyridyl)ethane
2-chlororesorcinol: tetramethylpyrazine: 2,2′-bipyridine: 4,4′-bipyridine
4,4′-bipyridine: methyl gallate: 2-chlororesorcinol: [2,2′-bipyridine]-4,4-diyldimethanol
4,4′-bipyridine: methyl gallate: 2-chlororesorcinol: [1,1′-biphenyl]-4,4′-diyldimethanol
4,4′-bipyridine: methyl gallate: 2-chlororesorcinol: 1,2-bis(4-pyridyl)ethane
4,4′-bipyridine: methyl gallate: 2-chlororesorcinol: 2,2′-bipyridine-4,4′-biphenylcarboxylic acid
4,4′-bipyridine: methyl gallate: 2-chlororesorcinol: tetramethylpyrazine
4,4′-bipyridine: methyl gallate: 2-chlororesorcinolcaffeine: 3,5-dinitrobenzoic acid: 2-methylresorcinol
Caffeine: 3,5-dinitrobenzoic acid: 2-methylresorcinol: 4,4′-dihydroxybiphenyl
Caffeine: 3,5-dinitrobenzoic acid: 2-methylresorcinol: orcinol
Caffeine: 3,5-dinitrobenzoic acid: 2-methylresorcinol: oxalic acid
resorcinol: tetramethylpyrazine: phenazine: pyrene

### Crystallisation Plate Readouts

After 7 days, the 96 well plates were examined by cross-polarised light microscopy. The results of the experiments in each well were classified as: F: fail (caused by a dispensing failure, resulting in no encapsulated droplet formation within the well); 1: remains in solution; 2: oiled-out or non-crystalline solid; 3: micro-crystalline solid; 4: crystals suitable for X-ray diffraction analysis (Figure S2).

Crystals analysed by SCXRD to be the desired co-crystal are given a * in the following plate readouts, and conditions are described in the footnote of the table. Crystals analysed by SCXRD where only unit cell data were collected are labelled alphabetically with a superscript and the structure is described in the footnote of the table. The plate readouts are provided in the tables below.

### Crystals Selected for SCXRD Analysis

Once crystals suitable for SCXRD analysis were obtained, the crystal of highest quality (most single and with the least number of defects) was selected. Droplets that provided the desired co-crystals (i.e. binary, ternary and quaternary co-crystals from binary, ternary and quaternary compound and co-former combinations, respectively) selected for X-ray diffraction analysis are summarised in Table 6.

TABLE 6
The compound(s) and co-former(s) solutions added to the droplet, the plate and
droplet position of the formed droplet, the solvent, oil and stock solution volume ratio present
in the droplet and the crystal structure (determined by SCXRD) of desired co-crystals suitable
for X-ray diffraction analysis.

| | Compound(s) | | | | Stock | |
|---|---|---|---|---|---|---|
| | and co- | Plate | | | solution | |
| | former(s) | and | | | volume | Crystal structure |
| No. | Binary | droplet | Solvent | Oil | ratio | (by SCXRD) |
| 1 | 4,4′-bipyridine: | P1 F4 | DMF | FC-40 | 2: 1 | 4,4′-bipyridine: |
| | methyl gallate | | | | | methyl gallate: H₂O: |
| | | | | | | DMF (3:2:2:1) |
| 2 | 4,4′-bipyridine: | P2 B2 | MeNO₂ | FC-40 | 2: 1 | 4,4′-bipyridine: |
| | methyl gallate | | | | | methyl gallate: |
| | | | | | | MeNO₂: H₂O |
| | | | | | | (1.5:1:1:1) |
| 3 | 4,4′-bipyridine: | P2 B7 | MeNO₂ | FC-40 | 1: 1 | 4,4′-bipyridine: 3,5- |
| | 3,5- | | | | | dinitrobenzoic acid |
| | dinitrobenzoic | | | | | (1:1) |
| | acid | | | | | |
| 4 | 4,4′-bipyridine: | P2 F7 | 1,4- | FC-40 | 1: 1 | 4,4′-bipyridine: |
| | quinol | | dioxane | | | quinol (1:0.5) |
| 5 | 4,4′-bipyridine: | P1 D12 | MeOH | MO | 1:2 | 4,4′-bipyridine: 3- |
| | 3-hydroxy-2- | | | | | hydroxy-2-napthoic |
| | napthoic acid | | | | | acid (0.5:1) |
| 6 | 4,4′-bipyridine: | P2 B11 | MeNO₂ | FC-40 | 1:2 | 4,4′-bipyridine: 3- |
| | 3-hydroxy-2- | | | | | hydroxy-2-napthoic |
| | napthoic acid | | | | | acid (1.5:1) |
| 7 | 4,4′-bipyridine: | P1 C8 | MeOH | FY | 1: 1 | 4,4′-bipyridine: |
| | glutaric acid | | | | | glutaric acid (2:2) |
| 8 | 4,4′-bipyridine: | P1 D8 | MeOH | MO | 1: 1 | 4,4′-bipyridine: 2,4- |
| | 2,4- | | | | | dihydroxybenzoic |
| | dihydroxybenzoic | | | | | acid (1:1) |
| | acid | | | | | |
| 9 | caffeine: methyl | P1 D8 | MeOH | MO | 1: 1 | caffeine: methyl |
| | gallate | | | | | gallate (1:1) |
| 10 | caffeine: 3,5- | P1 G7 | DMF | FY | 1: 1 | caffeine: 3,5- |
| | dinitrobenzoic | | | | | dinitrobenzoic acid |
| | acid | | | | | (2:2) |
| 11 | caffeine: quinol | P2 F11 | 1,4- | FC-40 | 1:2 | caffeine: quinol |
| | | | dioxane | | | (1:1.5) |
| 12 | caffeine: 3- | P1 A7 | MeOH | PDMSO | 1: 1 | caffeine: 3-hydroxy- |
| | hydroxy-2- | | | | | 2-napthoic acid |
| | napthoic acid | | | | | (1:1) |
| 13 | caffeine: glutaric | P1 A8 | MeOH | PDMSO | 1: 1 | caffeine: glutaric |
| | acid | | | | | acid (1:1) |
| 14 | caffeine: 2,4- | P1 A10 | MeOH | PDMSO | 1:2 | caffeine: 2,4- |
| | dihydroxybenzoic | | | | | dihydroxybenzoic |
| | acid | | | | | acid: H₂O (1:1:1) |
| 15 | nicotinamide: | P2 C6 | MeNO₂ | FY | 1: 1 | nicotinamide: |
| | methyl gallate | | | | | methyl gallate (1:1) |
| 16 | nicotinamide: | P1 C12 | MeOH | FY | 1:2 | nicotinamide: 3,5- |
| | 3,5- | | | | | dinitrobenzoic acid: |
| | dinitrobenzoic | | | | | MeOH (2:2:2) |
| | acid | | | | | |
| 17 | nicotinamide: | P1 D2 | MeOH | MO | 2: 1 | nicotinamide: quinol |
| | quinol | | | | | (2:0.5) |
| 18 | nicotinamide: 3- | P1 C7 | MeOH | FY | 1: 1 | nicotinamide: 3- |
| | hydroxy-2- | | | | | hydroxy-2-napthoic |
| | napthoic acid | | | | | acid (1:1) |
| 19 | nicotinamide: | P1 B12 | MeOH | FC-40 | 1:2 | nicotinamide: |
| | glutaric acid | | | | | glutaric acid (1:1) |
| 20 | nicotinamide: | P1 B8 | MeOH | FC-40 | 1: 1 | nicotinamide: 2,4- |
| | 2,4- | | | | | dihydroxybenzoic |
| | dihydroxybenzoi | | | | | acid: MeOH (1:1:1) |
| | c acid | | | | | |
| 21 | nicotinamide: | P2 A2 | MeNO₂ | PDMSO | 2: 1 | nicotinamide: 2,4- |
| | 2,4- | | | | | dihydroxybenzoic |
| | dihydroxybenzoic | | | | | acid (1:1) |
| | acid | | | | | |

Ternary

| | | | | | | |
|---|---|---|---|---|---|---|
| 22 | toluic acid: | P1 F1 | MeNO₂ | FC-40 | 2: 1: 1 | toluic acid: |
| | isonicotinamide: | | | | | isonicotinamide: |
| | 3,5- | | | | | 3,5-dinitrobenzoic |
| | dinitrobenzoic | | | | | acid (1:1:1) |
| | acid | | | | | |
| 23 | 4,4′-bipyridine: | P2 D6 | DMF | MO | 2: 1:2 | 4,4′-bipyridine: |
| | orcinol: | | | | | orcinol: phenazine |
| | phenazine | | | | | (1:1:0.5) |
| 24 | nicotinamide: | P2 D11 | DMF | FC-40 | 1: 2:2 | nicotinamide: |
| | fumaric acid: | | | | | fumaric acid: |
| | isoniazid | | | | | isoniazid (1:1:1) |
| 25 | tetramethylpyrazine: | P2 F5 | MeNO₂ | FC-40 | 2: 1:2 | tetramethylpyrazine: |
| | 2,2′- | | | | | 2,2′-bipyridine: 2- |
| | bipyridine: 2- | | | | | chlororesorcinol |
| | chlororesorcinol | | | | | (2(0.5):0.5:1) |
| 26 | tetramethylpyrazine: | P3 E10 | MeNO₂ | FY | 1: 1: 1 | tetramethylpyrazine: |
| | 2,2′- | | | | | 2,2′-bipyridine: 2- |
| | bipyridine: 2- | | | | | chlororesorcinol |
| | chlororesorcinol | | | | | (1:1:1) |
| 27 | 4,4′-bipyridine: | P1 A10 | MeOH | PDMSO | 1: 1:1 | 4,4′-bipyridine: |
| | methyl gallate: | | | | | methyl gallate: 2- |
| | 2- | | | | | chlororesorcinol: |
| | chlororesorcinol | | | | | H₂O (3:2:1:2) |
| 28 | caffeine: 3,5- | P3 E6 | MeNO₂ | PDMSO | 1: 1:1 | caffeine: 3,5- |
| | dinitrobenzoic | | | | | dinitrobenzoic acid: |
| | acid: 2- | | | | | 2-methylresorcinol: |
| | methylresorcinol | | | | | H₂O (1:1:2:1) |
| 29 | nicotinamide: | P3 E1 | MeNO₂ | PDMSO | 1: 1:1 | nicotinamide: 3,5- |
| | 3,5- | | | | | dinitrobenzoic acid: |
| | dinitrobenzoic | | | | | glutaric acid (1:1:1) |
| | acid: glutaric | | | | | |
| | acid | | | | | |
| 30 | nicotinamide: | P1 A6 | MeOH | FY | 1:2: 1 | nicotinamide: 3,5- |
| | 3,5- | | | | | dinitrobenzoic acid: |
| | dinitrobenzoic | | | | | tetramethylpyrazine: |
| | acid: | | | | | H₂O (1:1:0.5:1) |
| | tetramethylpyrazine | | | | | |
| 31 | nicotinamide: | P3 H12 | 1,4- | MO | 1: 1:1 | nicotinamide: |
| | quinol: benzoic | | Dioxane | | | quinol: benzoic acid |
| | acid | | | | | (1:0.5:1) |

Quaternary

| | | | | | | |
|---|---|---|---|---|---|---|
| 32 | 2- | P1 H2 | 1,4- | FC-40 | 1: 1: 1: 1 | 2-chlororesorcinol: |
| | chlororesorcinol: | | Dioxane | | | tetramethylpyrazine: |
| | tetramethylpyrazine: | | | | | 2,2′-bithiophene: |
| | 2,2′- | | | | | 1,2-bis(4- |
| | bithiophene: | | | | | pyridyl)ethane |
| | 1,2-bis(4- | | | | | (1:0.5:0.5:0.5) |
| | pyridyl)ethane | | | | | |
| 33 | resorcinol: | P1 G8 | 1,4- | FY | 1: 1: 1: 1 | resorcinol: |
| | tetramethylpyrazine: | | Dioxane | | | tetramethylpyrazine: |
| | phenazine: | | | | | phenazine: pyrene |
| | pyrene | | | | | (2:1:1:2) |
| 34 | 2- | P1 A6 | MeOH | PDMSO | 1: 1: 1: 1 | 2-chlororesorcinol: |
| | chlororesorcinol: | | | | | tetramethylpyrazine: |
| | tetramethylpyrazine: | | | | | 2,2′-bipyridine: |
| | 2,2′- | | | | | 1,2-bis(4- |
| | bipyridine: 1,2- | | | | | pyridyl)ethane |
| | bis(4- | | | | | (1:0.5:0.5:0.5) |
| | pyridyl)ethane | | | | | |
| 35 | 2- | P1 A10 | MeOH | FY | 1: 1: 1: 1 | 2-bromoresorcinol: |
| | bromoresorcinol: | | | | | tetramethylpyrazine: |
| | tetramethylpyrazine: | | | | | 2,2′-bipyridine: |
| | 2,2′- | | | | | 1,2-bis(4- |
| | bipyridine: 1,2- | | | | | pyridyl)ethane |
| | bis(4- | | | | | (1:0.5:0.5:0.5) |
| | pyridyl)ethane | | | | | |
| 36 | 2- | P1 A12 | MeOH | FY | 1: 1: 1: 1 | 2-chlororesorcinol: |
| | chlororesorcinol: | | | | | tetramethylpyrazine: |
| | tetramethylpyrazine: | | | | | 2,2′-bipyridine: |
| | 2,2′- | | | | | 4,4′-bipyridine |
| | bipyridine: 4,4′- | | | | | (1:0.5:0.5:0.5) |
| | bipyridine | | | | | |
| 37 | caffeine: 3,5- | P1 E2 | MeNO₂ | PDMSO | 1: 1: 1:1 | caffeine: 3,5- |
| | dinitrobenzoic | | | | | dinitrobenzoic acid: |
| | acid: 2- | | | | | 2-methylresorcinol: |
| | methylresorcinol: | | | | | orcinol |
| | orcinol | | | | | (1:1:0.25:1.75) |

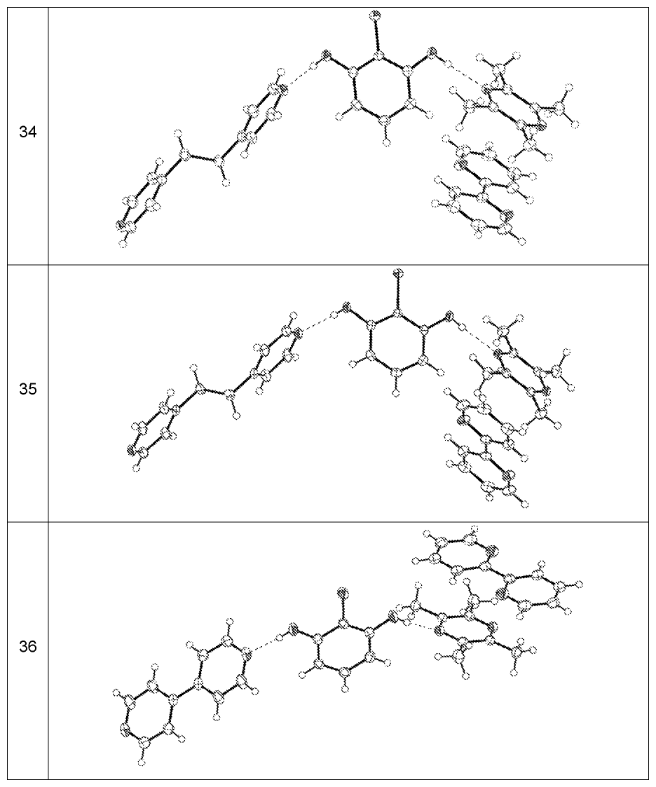

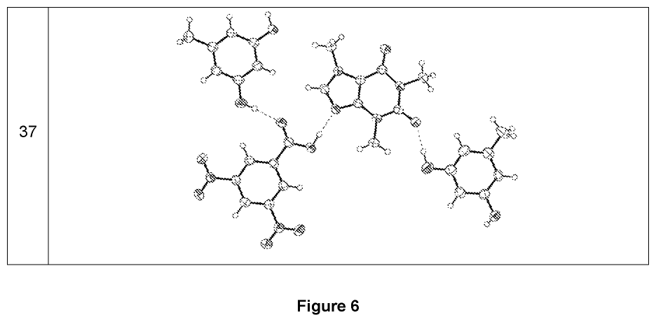

The crystal structures for each desired co-crystal (1 to 37) are shown in FIG. 6.

As demonstrated by the examples above, the method of the present invention can be used to form high quality co-crystals (e.g. binary, ternary and quaternary co-crystals) suitable for X-ray diffraction analysis from a range of chemical compounds and co-formers and using a range of solvents and oils. Co-crystals of suitable size and quality for XRPD analysis of every binary molecule/co-former combination tested in Example 6 was obtained. The method of the present invention also allowed co-crystals suitable for X-ray diffraction analysis to be formed that could not be formed without the presence of oil in the droplet.

### Example 7: Comparison of Method One Verses Crystallisation from Pre-Mixed Compound/Co-Former Solutions

In Method One (e.g. used in Example 6, above) the appropriate volume of compound stock solution is picked up, followed by the sequential pick-up of the appropriate volume of one or more co-former stock solutions into the same needle. The solutions are then injected into the oil droplet. The efficacy of such a method was assessed by comparing the results obtained for the binary co-crystallisation experiments in Example 6 to a method in which the compound and co-former solutions were mixed prior to injection into the oil droplet, (i.e. Method Four, described in further detail below).

For all the binary combination systems shown in Table 5, above, in 2-mL screw top vial, stock solutions of both compound and co-formers were prepared separately by dissolving each compound in a solvent to give the concentrations as shown in Table 1. Mixed solutions of each compound and co-former combination were then prepared by mixing the compound and co-former stock solutions in the appropriate (2:1, 1:1, and 1:2) ratios.

The appropriate volume of these mixed stock solutions was then injected into the oil droplet according to the plate layouts as shown in Table 2. After 7 days, crystallisation outcomes were assessed by cross-polarised optical microscopy and classified as “single crystals suitable for SCXRD”, “microcrystalline”, “non-crystalline solids or oils”, “remaining in solution”, and compared to outcomes in Example 6.

Method One gave improved outcomes for all three compounds (4,4′-bipyridine, caffeine and nicotinamide) in combination with the selected co-formers when compared to the use of mixed stock solutions, as judged by the propensity to form “single crystals suitable for SCXRD”, for 4,4′-bipyridine 29% vs 23%, for caffeine 28% vs 5% and for nicotinamide 24% vs 6%.

1. A method of forming a co-crystal comprising a chemical molecule, or a salt thereof, and a co-former, or a salt thereof, the method comprising:
a1) obtaining a first solution, said first solution comprising the chemical molecule, or the salt thereof, dissolved in an organic medium;
a2) obtaining a second solution, said second solution comprising the co-former, or the salt thereof, dissolved in an organic medium;
b) forming a droplet, the droplet comprising a first liquid and a second liquid; wherein the first liquid comprises a mixture of the first and second solutions; and the second liquid is an oil; and
c) allowing the co-crystal comprising the chemical molecule, or the salt thereof, and the co-former, or the salt thereof, to form in the droplet.

2. A method of claim 1, wherein the chemical molecule is an organic molecule.

3. A method of claim 1 or claim 2, wherein the chemical molecule is a small molecule.

4. A method of any one of claims 1 to 3, wherein each organic medium comprises at least one organic solvent.

5. A method of claim 4, wherein the total amount of organic solvent in each organic medium is greater than 75% by volume of the organic medium.

6. A method of any one of claims 1 to 5, wherein at least one organic solvent in each organic medium has a boiling point greater than 80° C.

7. A method of claim 6, wherein at least one organic solvent in each organic medium has a boiling point greater than 125° C.

8. A method of any one of claims 1 to 7, wherein the oil is a perfluorinated oil, a paraffin oil, or a silicon oil.

9. A method of any one of claims 1 to 8, wherein in the droplet, the second liquid forms a coating over the first liquid.

10. A method of claim 9, wherein the second liquid encapsulates the first liquid.

11. A method of any one of claims 1 to 10, wherein the ratio first liquid:second liquid is in the range from 2:1 to 1:20 by volume.

12. A method of any one of claims 1 to 11, wherein the droplet is a sitting droplet.

13. A method of any one of claims 1 to 12, wherein the droplet is a hanging droplet.

14. A method of any one of claims 1 to 13, wherein the surface on which the droplet is formed is a well of a multi-well plate.

15. A method of any one of claims 1 to 14, wherein the method further comprises step a3): obtaining a third solution, said third solution comprising a second co-former or a salt thereof dissolved in an organic medium; wherein the first liquid comprises a mixture of the first, second and third solutions and wherein the co-crystal formed in step c) comprises the chemical molecule and both co-formers (with each component being optionally present as a salt thereof).

16. A method of any one of claims 1 to 15, wherein the organic medium in the first solution and the second solution are the same.

17. A method of any one of claims 1 to 15, wherein the organic medium in the first solution and the second solution are different.

18. A method of any one of claims 1 to 17, wherein step b) comprises: forming a droplet of the second liquid; taking the first solution and the second solution up into a syringe to form the first liquid in the syringe; and injecting the first liquid into the second liquid to form the droplet.

19. A method of any one of claims 1 to 17, wherein step b) comprises: taking the first solution and the second solution up into a syringe to form the first liquid in the syringe;
forming a droplet of the first liquid; and coating the second liquid onto the droplet of the first liquid to form the droplet.

20. A method of any one of claims 1 to 17, wherein step b) comprises: taking the first solution and the second solution up into separate syringes; mixing the solutions to form a droplet of the first liquid; and coating the second liquid onto the droplet of the first liquid to form the droplet.

21. A method of any one of claims 1 to 17, wherein step b) comprises: forming a droplet of the second liquid; taking the first solution and the second solution up into separate syringes; and injecting the first solution and the second solution separately into the second liquid to form the droplet.

22. A method of screening for co-crystals of a chemical molecule, or salt thereof, the method comprising:
forming a plurality of droplets according to the method of any one of claims 1 to 21;
allowing crystals to form from said plurality of droplets;
optionally looking for signs of crystal formation;
recovering any co-crystals that are formed;
performing single crystal X-ray crystallography on the co-crystals that are formed; and
optionally, comparing the results of the single crystal X-ray crystallography for each co-crystal to the results of the single crystal X-ray crystallography for each other crystal and/or to known co-crystals of the chemical (e.g. organic) molecule, or salt thereof;
wherein at least two of the plurality of droplets are different.

23. A method of claim 22, wherein each droplet is formed in a respective well of a multiwell plate.

24. A method of 22 or claim 23, wherein the plurality of droplets may each be different.
